(19) United States
(12) Patent Application Publication
(10) Pub. No.: US 2015/0337002 A1
(43) Pub. Date: Nov. 26, 2015

(54) NUCLEOSIDE AND NUCLEOTIDE HAVING SULFONAMIDE STRUCTURE

(71) Applicants: OSAKA UNIVERSITY, Osaka (JP); SHIONOGI & CO., LTD., Osaka-shi (JP)

(72) Inventors: Satoshi OBIKA, Suita-shi (JP); Yasunori MITSUOKA, Toyonaka-shi (JP)

(73) Assignees: SHIONOGI & CO., LTD., Osaka-shi (JP); OSAKA UNIVERSITY, Suita-shi (JP)

(21) Appl. No.: 14/759,099

(22) PCT Filed: Jan. 14, 2014
(86) PCT No.: PCT/JP2014/050429

§ 371 Date: Jul. 2, 2015

(30) Foreign Application Priority Data
Jan. 15, 2013 (JP) 2013-004735

(51) Int. Cl. C07H 19/10; C07H 19/20

(52) U.S. Cl. CPC C07H 19/10; C07H 19/20

(57) ABSTRACT

The present invention provides compounds shown by the formula:

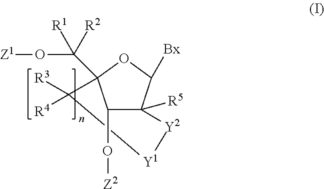

wherein
Y¹-Y² is S(═O)₂—NR⁶, NR⁶—S(═O)₂ or the like,
R⁶ is a hydrogen atom, substituted or unsubstituted alkyl or the like,
Bx is a nucleic acid base moiety,
Z¹ and Z² are each independently, a hydrogen atom, a hydroxyl protecting group or a reactive phosphorus group,
R¹ to R⁵ are each independently, a hydrogen atom, halogen, cyano, substituted or unsubstituted alkyl or the like, and
n is an integer of 0 to 3,
or salts thereof, that are novel nucleosides or nucleotides that can be useful as materials for synthesizing nucleic acid pharmaceuticals.

### FIELD OF THE INVENTION

The present invention relates to a novel bridged nucleoside or nucleotide. In more detail, it relates to a nucleoside or nucleotide having a bridge comprising a sulfonamide structure, or an oligonucleotide prepared with the nucleoside(s) or nucleotide(s).

### BACKGROUND ART

As a therapy for a disease with a nucleic acid pharmaceutical, there is a method with an antisense oligonucleotide, siRNA, ribozyme, antigene, aptamer, decoy nucleic acid or the like.

An antisense oligonucleotide is an oligonucleotide complementary to mRNA, mRNA precursor or ncRNA (non-coding RNA), such as ribosomal RNA, transfer RNA, miRNA and the like, of the target gene, and a single strand DNA, RNA and/or structural analog thereof which consists of about 8 to 30 bases. The antisense oligonucleotide suppresses the function of mRNA, mRNA precursor or ncRNA by forming a double strand with the target mRNA, mRNA precursor or ncRNA.

A siRNA is a low molecular weight double-strand RNA complementary to the target gene which consists of about 19 to 25 base pairs. It relates to a phenomenon called RNA interference, and suppresses the gene expression by base sequence-specific mRNA degradation.

A ribozyme is RNA with enzyme activity of cleaving a nucleic acid. It cleaves specifically the mRNA of the target gene by forming double strands with the mRNA.

An antigene is an oligonucleotide corresponding to a double strand DNA moiety of the target gene. It suppresses transcription from the DNA to mRNA by forming triple strands with the DNA moiety and oligonucleotide.

An aptamer is a DNA, RNA and/or structural analog thereof which specifically bonds to a specific molecule. It inhibits the function of the target protein by binding to the protein.

A decoy nucleic acid is a short DNA comprising the same sequence with a binding site for a specific transcription modulating factor. It inhibits binding with the transcription modulating factor and gene, and suppresses expression of the gene groups activated by the transcription modulating factor.

Various nucleosides or nucleotides are developed as materials for synthesizing the above nucleic acid pharmaceuticals. Examples include S-oligo (phosphorothioate) which is modified the phosphate moiety of a nucleotide, 2′,4′-BNA (bridged nucleic acid)/LNA (locked nucleic acid) which is modified the sugar moiety of a nucleoside or nucleotide (Patent Documents 1 to 5 and Non-patent Documents 1 to 6) and the like.

### PRIOR ART DOCUMENT

### Patent Document

[Patent Document 1] WO98/39352
[Patent Document 2] WO2005/021570
[Patent Document 3] WO2003/068795
[Patent Document 4] WO2011/052436
[Patent Document 5] WO2011/156202

### Non-Patent Document

[Non-patent Document 1] Proc. Natl. Acad. Sci. USA, 2000, vol. 97, no. 10, 5633-5638
[Non-patent Document 2] Bioorg. Med. Chem., 2006, vol. 14, 1029-1038
[Non-patent Document 3] Chem. Commun., 2007, 3765-3767
[Non-patent Document 4] J. Am. Chem. Soc., 2008, vol. 130, no. 14, 4886-4896
[Non-patent Document 5] Nucleic Acids Res., 2008, vol. 36, no. 13, 4257-4265
[Non-patent Document 6] Bioorg. Med. Chem., 2001, vol. 9, 1001-1011

### SUMMARY OF THE INVENTION

### Problems to be Solved by the Invention

The present invention is intended to provide novel nucleosides or nucleotides that can be useful as materials for synthesizing nucleic acid pharmaceuticals such as antisense oligonucleotides, siRNAs, ribozymes, antigenes, aptamers, decoy nucleic acids and the like.

### Means for Solving the Problem

The present inventors have intensively studied to synthesize novel bridged nucleosides or nucleotides with the superior binding affinity to a single strand RNA and nuclease resistance. The nucleosides or nucleotides are useful very much as materials for synthesizing nucleic acid pharmaceuticals such as antisense oligonucleotides and the like.

That is, the present invention relates to the following.

(1) A compound of formula (I) or a salt thereof:

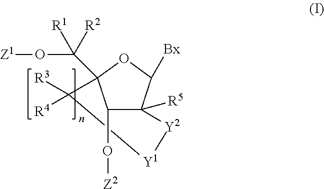

wherein

### Y¹-Y² is S(═O)—NR⁶, S(═O)₂—NR⁶, NR⁶—S(═O) or NR⁶—S(═O)₂,

R⁶ is a hydrogen atom, substituted or unsubstituted alkyl, substituted or unsubstituted alkenyl, or substituted or unsubstituted alkynyl,
Bx is a nucleic acid base moiety,
Z¹ and Z² are each independently, a hydrogen atom, a hydroxyl protecting group or a reactive phosphorus group,
R¹ and R² are each independently, a hydrogen atom, halogen, cyano, substituted or unsubstituted alkyl, substituted or unsubstituted alkenyl, or substituted or unsubstituted alkynyl,
R³ are each independently, a hydrogen atom, halogen, cyano, substituted or unsubstituted alkyl, substituted or unsubstituted alkenyl, or substituted or unsubstituted alkynyl,
R⁴ are each independently, a hydrogen atom, halogen, cyano, substituted or unsubstituted alkyl, substituted or unsubstituted alkenyl, or substituted or unsubstituted alkynyl,
R⁵ is a hydrogen atom, halogen, cyano, substituted or unsubstituted alkyl, substituted or unsubstituted alkenyl, or substituted or unsubstituted alkynyl, and
n is an integer of 0 to 3.
(2) The compound or salt thereof of (1), wherein Y¹-Y² is S(═O)₂—NR⁶, and R⁶ is a hydrogen atom or alkyl.
(3) The compound or salt thereof of (1) or (2), wherein Bx is substituted or unsubstituted purin-9-yl, or substituted or unsubstituted 2-oxo-pyrimidin-1-yl.
(4) The compound or salt thereof of any one of (1) to (3), wherein Z¹ is a hydrogen atom or a hydroxyl protecting group.
(5) The compound or salt thereof of (4), wherein the hydroxyl protecting group is acetyl, t-butyl, t-butoxymethyl, methoxymethyl, tetrahydropyranyl, 1-ethoxyethyl, 1-(2-chloroethoxy)-ethyl, 2-trimethylsilylethyl, p-chlorophenyl, 2,4-dinitrophenyl, benzyl, benzoyl, p-phenylbenzoyl, 2,6-dichlorobenzyl, levulinoyl, diphenylmethyl, p-nitrobenzyl, trimethylsilyl, triethylsilyl, t-butyldimethylsilyl, t-butyldiphenylsilyl, triphenylsilyl, triisopropylsilyl, benzoyl formate, chloroacetyl, trichloroacetyl, trifluoroacetyl, pivaloyl, isobutyryl, 9-fluorenylmethyloxycarbonyl, methansulfonyl, p-toluenesulfonyl, trifluoromethanesulfonyl, trityl, monomethoxytrityl, dimethoxytrityl, trimethoxytrityl, 9-phenylxanthin-9-yl or 9-(p-methoxyphenyl) xanthin-9-yl.
(6) The compound or salt thereof of any one of (1) to (5), wherein Z² is a hydrogen atom or a reactive phosphorus group.
(7) The compound or salt thereof of (6), wherein the reactive phosphorus group is diisopropylcyanoethoxy phosphoramidite or H-phosphonate.
(8) An oligonucleotide comprising one or more nucleoside structure of formula (II) or a pharmaceutically acceptable salt thereof:

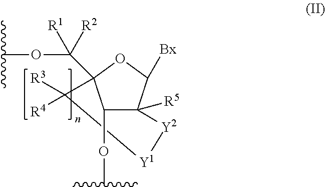

wherein

### Y¹-Y² is S(═O)—NR⁶, S(═O)₂—NR⁶, NR⁶—S(═O) or NR⁶—S(═O)₂,

R⁶ is a hydrogen atom, substituted or unsubstituted alkyl, substituted or unsubstituted alkenyl, or substituted or unsubstituted alkynyl,
Bx is a nucleic acid base moiety,
R¹ and R² are each independently, a hydrogen atom, halogen, cyano, substituted or unsubstituted alkyl, substituted or unsubstituted alkenyl, or substituted or unsubstituted alkynyl,
R³ are each independently, a hydrogen atom, halogen, cyano, substituted or unsubstituted alkyl, substituted or unsubstituted alkenyl, or substituted or unsubstituted alkynyl,
R⁴ are each independently, a hydrogen atom, halogen, cyano, substituted or unsubstituted alkyl, substituted or unsubstituted alkenyl, or substituted or unsubstituted alkynyl,
R⁵ is a hydrogen atom, halogen, cyano, substituted or unsubstituted alkyl, substituted or unsubstituted alkenyl, or substituted or unsubstituted alkynyl, and
n is an integer of 0 to 3.

### Effect of the Invention

An oligonucleotide prepared with the nucleotide(s) or nucleoside(s) of the present invention shows the superior binding affinity to a single strand RNA and nuclease resistance. The oligonucleotide is thought to have very good persistence in vivo, and therefore expected to apply to nucleic acid pharmaceuticals.

### BRIEF DESCRIPTION OF THE DRAWINGS

FIG. 1 A graph showing the exonuclease resistant properties of the oligonucleotides of the present invention. (Example 4)

### EMBODIMENTS FOR CARRYING OUT THE INVENTION

Terms used herein, unless otherwise indicated, are used in a sense normally used in the art.

Terms used in this description are explained below. Each term, unless otherwise indicated, has the same meaning when it is used alone or together with other terms.

The term “nucleic acid base moiety” means a substituent containing a nucleic acid base or an analog thereof. Examples of natural nucleic acid bases include adenine (A), guanine (G), thymine (T), cytosine (C) and uracil (U). The nucleic acid base of the present invention is not limited to them, and includes the other artificial or natural nucleic acid bases. Examples include 5-methylcytosine (5-me-C), 5-hydroxymethylcytosine, xanthine, hypoxanthine, 2-aminoadenine, 7-deaza-adenine, 7-deazaguanosine, 2-aminopyridine, 2-pyridone and the like.

In other words, the “nucleic acid base moiety” for the present invention is substituted or unsubstituted heterocyclyl, or substituted or unsubstituted carbocyclyl which constitutes a base moiety of a nucleic acid (DNA or RNA).

The heterocycle includes monocycle or polycycle, containing one or more of heteroatom(s) selected independently from O, S and N. Examples include purine, pyrimidine, thiophene, thianthrene, furan, pyran, isobenzofuran, chromene, xanthene, phenoxathiin, pyrrole, imidazole, pyrazole, isothiazole, isoxazole, pyridazine, indolizine, indole, isoindole, isoquinoline, quinoline, naphthyridine, quinoxaline, quinazoline, pteridine, carbazole, phenanthridine, acridine, perimidine, phenazine, phenarsazine, phenothiazine, furazan, phenoxazine, pyrrolidine, pyrroline, imidazolidine, imidazoline, pyrazolidine and the like. Preferably it is purine or pyrimidine.

The carbocycle includes monocyclic or polycyclic hydrocarbocycle. Examples include benzene, naphthalene, anthracene, phenanthrene, indane, indene, tetrahydronaphthalene, biphenylene and the like. Preferably, it is benzene or naphthalene.

The substituent for the heterocyclyl or carbocyclyl is a substituent selected from Substituent group α. A carbon atom at any position may bind to one or more substituent(s) selected from Substituent group α.

Substituent group α: halogen, hydroxy, a hydroxyl group protected with a protective group for synthesis of nucleic acid, alkyl, alkyloxy, alkylthio, alkylamino, alkenyl, alkynyl, mercapto, a mercapto group protected with a protective group for synthesis of nucleic acid, amino, and an amino group protected with a protective group for synthesis of nucleic acid.

The protective group for “a hydroxyl group protected with a protective group for synthesis of nucleic acid” is not limited, as long as it can stably protect a hydroxyl group during synthesis of nucleic acid. Concretely, it is a protective group which is stable under acidic or neutral conditions and which can be cleft by a chemical method such as hydrogenolysis, hydrolysis, electrolysis or photolysis. Examples include substituted or unsubstituted alkyl, substituted or unsubstituted alkenyl, formyl or the following protective groups.

Aliphatic acyl: alkylcarbonyl such as acetyl, propionyl, butyryl, isobutyryl, pentanoyl, pivaloyl, valeryl, isovaleryl, octanoyl, nonanoyl, decanoyl, 3-methylnonanoyl, 8-methylnonanoyl, 3-ethyloctanoyl, 3,7-dimethyloctanoyl, undecanoyl, dodecanoyl, tridecanoyl, tetradecanoyl, pentadecanoyl, hexadecanoyl, 1-methylpentadecanoyl, 14-methylpentadecanoyl, 13,13-dimethyltetradecanoyl, heptadecanoyl, 15-methylhexadecanoyl, octadecanoyl, 1-methylheptadecanoyl, nonadecanoyl, eicosanoyl, heneicosanoyl and the like, carboxylated alkylcarbonyl such as succinoyl, glutaroyl, adipoyl and the like, haloalkylcarbonyl such as chloroacetyl, dichloroacetyl, trichloroacetyl, trifluoroacetyl and the like, alkyloxyalkylcarbonyl such as methoxyacetyl and the like, and unsaturated alkylcarbonyl such as (E)-2-methyl-2-butenoyl and the like, etc.
Aromatic acyl: arylcarbonyl such as benzoyl, α-naphthoyl, β-naphthoyl and the like, halogenoarylcarbonyl such as 2-bromobenzoyl, 4-chlorobenzoyl and the like, alkylated arylcarbonyl such as 2,4,6-trimethylbenzoyl, 4-toluoyl and the like, alkyloxylated arylcarbonyl such as 4-anisoyl and the like, carboxylated arylcarbonyl such as 2-carboxybenzoyl, 3-carboxybenzoyl, 4-carboxybenzoyl and the like, nitrated arylcarbonyl such as 4-nitrobenzoyl, 2-nitrobenzoyl and the like, alkyloxycarbonylated arylcarbonyl such as 2-(methoxycarbonyl)benzoyl and the like, and arylated arylcarbonyl such as 4-phenylbenzoyl and the like, etc.
Tetrahydropyranyl: tetrahydropyran-2-yl, 3-bromotetrahydropyran-2-yl, 4-methoxytetrahydropyran-4-yl and the like.
Tetrahydrothiopyranyl: tetrahydrothiopyran-2-yl, 4-methoxytetrahydrothiopyran-4-yl and the like.
Tetrahydrofuranyl: tetrahydrofuran-2-yl and the like.
Tetrahydrothiofuranyl: tetrahydrothiofuran-2-yl and the like.
Silyl: trialkylsilyl such as trimethylsilyl, triethylsilyl, isopropyldimethylsilyl, t-butyldimethylsilyl, methyldiisopropylsilyl, methyl di-t-butylsilyl, triisopropylsilyl and the like, trialkylsilyl substituted by 1 or 2 aryl, such as diphenylmethylsilyl, diphenylbutylsilyl, diphenylisopropylsilyl, phenyldiisopropylsilyl and the like, etc.
Alkyloxymethyl: methoxymethyl, 1,1-dimethyl-1-methoxymethyl, ethoxymethyl, propoxymethyl, isopropoxymethyl, butoxymethyl, t-butoxymethyl and the like.
Alkyloxylated alkyloxymethyl: 2-methoxyethoxymethyl and the like.
Halogeno alkyloxymethyl: 2,2,2-trichloroethoxymethyl, bis(2-chloroethoxy)methyl and the like.
Alkyloxylated ethyl: 1-ethoxyethyl, 1-(isopropoxy)ethyl and the like.
Halogenated ethyl: 2,2,2-trichloroethyl and the like.
Methyl substituted by 1 to 3 aryl: benzyl, α-naphthylmethyl, β-naphthylmethyl, diphenylmethyl, triphenylmethyl, α-naphthyldiphenylmethyl, 9-anthrylmethyl and the like.
Methyl substituted by 1 to 3 aryl, with the aryl ring being substituted by alkyl, alkyloxy, halogen or cyano: 4-methylbenzyl, 2,4,6-trimethylbenzyl, 3,4,5-trimethylbenzyl, 4-methoxybenzyl, 4-methoxyphenyldiphenylmethyl, 4,4′-dimethoxytriphenylmethyl, 2-nitrobenzyl, 4-nitrobenzyl, 4-chlorobenzyl, 4-bromobenzyl, 4-cyanobenzyl and the like.
Alkyloxycarbonyl: methoxycarbonyl, ethoxycarbonyl, t-butoxycarbonyl, isobutoxycarbonyl and the like.
Aryl substituted by halogen, alkyloxy or nitro: 4-chlorophenyl, 2-fluorophenyl, 4-methoxyphenyl, 4-nitrophenyl, 2,4-dinitrophenyl and the like.
Alkyloxycarbonyl substituted by halogen or trialkylsilyl: 2,2,2-trichloroethoxycarbonyl, 2-trimethylsilylethoxycarbonyl and the like.
Alkenyloxycarbonyl: vinyloxycarbonyl, aryloxycarbonyl and the like.
Aralkyloxycarbonyl having an aryl ring optionally substituted by 1 or 2 alkyloxy or nitro: benzyloxycarbonyl, 4-methoxybenzyloxycarbonyl, 3,4-dimethoxybenzyloxycarbonyl, 2-nitrobenzyloxycarbonyl, 4-nitrobenzyloxycarbonyl and the like.

A preferable protective group is alkyl, alkenyl, “aliphatic acyl”, “aromatic acyl”, “methyl substituted by 1 to 3 aryl”, “aryl substituted by halogen, alkyloxy or nitro” or the like. More preferably, it is benzoyl, benzyl, 2-chlorophenyl, 4-chlorophenyl, 2-propenyl or the like.

The protective group for “a mercapto group protected with a protective group for synthesis of nucleic acid” is not limited, as long as it can stably protect a mercapto group during synthesis of nucleic acid. Concretely, it is a protective group which is stable under acidic or neutral conditions and which can be cleft by a chemical method such as hydrogenolysis, hydrolysis, electrolysis or photolysis.

Examples include not only those named above as the protective group for a hydroxyl group, but also the following.

Disulfide-forming groups: alkylthio such as methylthio, ethylthio, tert-butylthio and the like, arylthio such as benzylthio and the like, etc.

A preferable protective group is “aliphatic acyl”, “aromatic acyl” and the like. More preferably, it is benzoyl or the like.

The protective group for “amino protected with a protective group for synthesis of nucleic acid” is not limited, as long as it can stably protect an amino group during synthesis of nucleic acid. Concretely, it is a protective group which is stable under acidic or neutral conditions and which can be cleft by a chemical method such as hydrogenolysis, hydrolysis, electrolysis or photolysis.

Examples include formyl and the above protective groups for the hydroxyl group, such as “aliphatic acyl”, “aromatic acyl”, “alkyloxycarbonyl”, “alkyloxycarbonyl substituted by halogen or trialkylsilyl”, “alkenyloxycarbonyl” and “aralkyloxycarbonyl having an aryl ring optionally substituted by 1 or 2 alkyloxy or nitro”.

A preferable protective group is “aliphatic acyl”, “aromatic acyl” or the like. More preferably, it is benzoyl or the like.

The “nucleic acid base moiety” is preferably substituted or unsubstituted purin-9-yl, substituted or unsubstituted 2-oxo-pyrimidin-1-yl or the like. The substituent for a ring containing a nucleic acid base moiety is a substituent selected from the above Substituent group α. A carbon atom at any position may bind to one or more substituent(s) selected from Substituent group α. More preferably, it is purin-9-yl or 2-oxo-pyrimidin-1-yl substituted by one or more substituent(s) selected from the above Substituent group α. Especially preferably, it is purin-9-yl or 2-oxo-pyrimidin-1-yl substituted by 1 or 2 substituent(s) selected from the above Substituent group α.

Examples include 6-aminopurin-9-yl (i.e. adeninyl), 6-aminopurin-9-yl having an amino protected with a protective group for synthesis of nucleic acid, 2,6-diaminopurin-9-yl, 6-chloropurin-9-yl, 2-amino-6-chloropurin-9-yl, 2-amino-6-chloropurin-9-yl having an amino protected with a protective group for synthesis of nucleic acid, 6-fluoropurin-9-yl, 2-amino-6-fluoropurin-9-yl, 2-amino-6-fluoropurin-9-yl having an amino protected with a protective group for synthesis of nucleic acid, 6-bromopurin-9-yl, 2-amino-6-bromopurin-9-yl, 2-amino-6-bromopurin-9-yl having an amino protected with a protective group for synthesis of nucleic acid; 2-amino-6-hydroxypurin-9-yl (i.e. guaninyl), 2-amino-6-hydroxypurin-9-yl having an amino protected with a protective group for synthesis of nucleic acid, 6-amino-2-methoxypurin-9-yl, 6-amino-2-chloropurin-9-yl, 6-amino-2-fluoropurin-9-yl, 2,6-dimethoxypurin-9-yl, 2,6-dichloropurin-9-yl, 6-mercaptopurin-9-yl, 2-oxo-4-amino-1,2-dihydropyrimidin-1-yl (i.e. cytosinyl), 2-oxo-4-amino-1,2-dihydropyrimidin-1-yl having an amino protected with a protective group for synthesis of nucleic acid, 2-oxo-4-amino-5-fluoro-1,2-dihydropyrimidin-1-yl, 2-oxo-4-amino-5-fluoro-1,2-dihydropyrimidin-1-yl having an amino protected with a protective group for synthesis of nucleic acid, 4-amino-2-oxo-5-chloro-1,2-dihydropyrimidin-1-yl, 2-oxo-4-methoxy-1,2-dihydropyrimidin-1-yl, 2-oxo-4-mercapto-1,2-dihydropyrimidin-1-yl, 2-oxo-4-hydroxy-1,2-dihydropyrimidin-1-yl (i.e. uracinyl), 2-oxo-4-hydroxy-5-methyl-1,2-dihydropyrimidin-1-yl (i.e. thyminyl), 4-amino-5-methyl-2-oxo-1,2-dihydropyrimidin-1-yl (i.e. 5-methyl cytosinyl), 4-amino-5-methyl-2-oxo-1,2-dihydropyrimidin-1-yl having an amino protected with a protective group for synthesis of nucleic acid and the like.

More concretely, groups of formula: (B-1) to (B-4) described below are exemplified.

A group of formula (B-1):

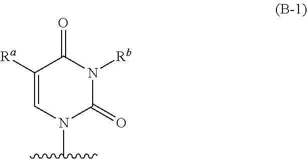

wherein
R^{a} is a hydrogen atom or alkyl, and
R^{b} is a hydrogen atom or alkyl.

R^{a} is preferably a hydrogen atom or C1 to C5 alkyl. A hydrogen atom or methyl is more preferable.

R^{b} is preferably a hydrogen atom.

A group of formula (B-2):

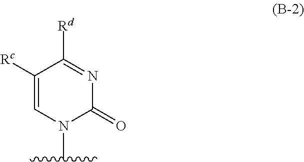

wherein
R^{c} is a hydrogen atom, halogen or alkyl,
R^{d} is amino, mercapto, alkyloxy, NHCOR^{e}, NHCOCH₂OR^{e} or N═NR^{f},
R^{e} is substituted or unsubstituted alkyl, or substituted or unsubstituted aromatic carbocyclyl, and
R^{f} is a hydrogen atom or alkyl.

R^{e} is preferably a hydrogen atom or C1 to C5 alkyl. A hydrogen atom or methyl is more preferable.

R^{d} is preferably NHCOPh, NHCOCH₃, NHCOCH₂OPh or NHCOCH₂O-(4-tBu)Ph.

A group of formula (B-3):

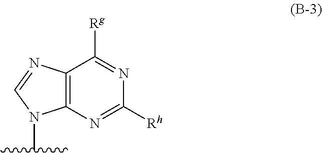

wherein
R^{g} is halogen, amino, mercapto, alkyloxy, NHCORⁱ, NHCOCH₂ORⁱ or N═NR^{j}),
R^{h} is a hydrogen atom, halogen, amino or alkyloxy,
Rⁱ is substituted or unsubstituted alkyl, or substituted or unsubstituted aromatic carbocyclyl, and
R^{j} is a hydrogen atom or alkyl.

R^{g} is preferably NHCOPh, NHCOCH₃, NHCOCH₂OPh or NHCOCH₂O-(4-tBu)Ph.

R^{h} is preferably a hydrogen atom.

A group of formula (B-4):

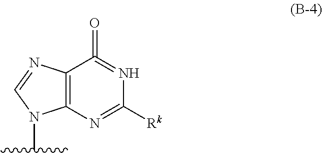

wherein
R^{k} is amino, NHCOR^{m}, NHCOCH₂OR^{m} or N═NRⁿ,
R^{m} is substituted or unsubstituted alkyl, or substituted or unsubstituted aromatic carbocyclyl, and
Rⁿ is a hydrogen atom or alkyl.

R^{k} is preferably NHCOPh, NHCOCH₃, NHCOCH(CH₃)₂, NHCOCH₂OPh or NHCOCH₂O-(4-tBu)Ph.

Examples include as follows.

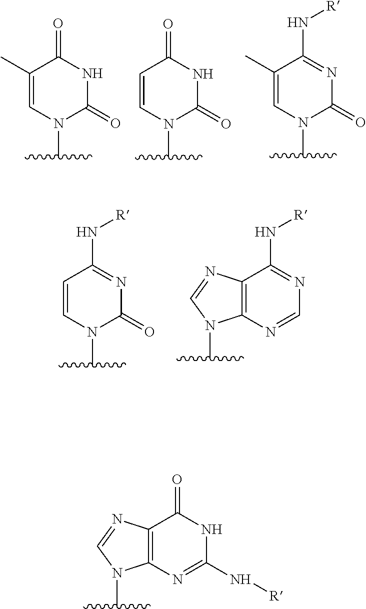

wherein R′ is a hydrogen atom or a protecting group for amino used in nucleic acid synthesis. Examples include isobutyl, acetyl, benzoyl, phenoxyacetyl and the like.

The term “hydroxyl protecting group” for Z¹ and Z² includes those named above as “a hydroxyl group protected with a protective group for synthesis of nucleic acid”. Preferably, it is alkyl, alkenyl, “aliphatic acyl”, “aromatic acyl” or the like.

More preferably, it is acetyl, t-butyl, t-butoxymethyl, methoxymethyl, tetrahydropyranyl, 1-ethoxyethyl, 1-(2-chloroethoxyl)ethyl, 2-trimethylsilylethyl, p-chlorophenyl, 2,4-dinitrophenyl, benzyl, benzoyl, p-phenylbenzoyl, 2,6-dichlorobenzyl, levulinoyl, diphenylmethyl, p-nitrobenzyl, trimethylsilyl, triethylsilyl, t-butyldimethylsilyl, t-butyldiphenylsilyl, triphenylsilyl, triisopropylsilyl, benzoyl formate, chloroacetyl, trichloroacetyl, trifluoroacetyl, pivaloyl, isobutyryl, 9-fluorenylmethyloxycarbonyl, methansulfonyl, p-toluenesulfonyl, trifluoromethanesulfonyl, triphenylmethyl (trityl), monomethoxytrityl, dimethoxytrityl (DMTr), trimethoxytrityl, 9-phenylxanthin-9-yl (Pixyl) or 9-(p-methoxyphenyl) xanthin-9-yl (MOX). Especially preferably, it is benzyl, triethylsilyl, t-butyldimethylsilyl, t-butyldiphenylsilyl, triisopropylsilyl, trityl, monomethoxytrityl, dimethoxytrityl, trimethoxytrityl or the like.

The term “reactive phosphorus group” means a group containing phosphorous atom(s) which is (are) useful for forming an internucleoside linkage such as phosphodiester and phosphorotioate internucleoside linkages. The reactive phosphorous groups publicly known in the art can be used and examples include phosphoramidite, H-phosphonate, phosphate diesters, phosphate triesters, phosphorus containing chiral auxiliaries and the like.

Concretely, examples include groups of the following formula: (Z²-1) to (Z²-3).

A group of formula (Z²-1): —P(OR^{X1})(NR^{X2}) wherein R^{X1} is substituted or unsubstituted alkyl and R^{X2} is substituted or unsubstituted alkyl. R^{X1} is preferably alkyl or cyanoalkyl. R^{X2} is preferably alkyl.

A group of formula (Z²-2): —P(═R^{X3})(OR^{X4})₂ wherein R^{X3} is O or S, and R^{X4} are each independently, a hydrogen atom, a protective group used in nucleic acid synthesis, substituted or unsubstituted alkyl, or substituted or unsubstituted aromatic carbocyclyl. R^{X3} is preferably O, and R^{X4} is preferably a hydrogen atom.

A group of formula (Z²-3): —P(═R^{X5})H(OR^{X6}) wherein R^{X5} is O or S, and R^{X6} is a hydrogen atom, a protective group used in nucleic acid synthesis, or substituted or unsubstituted aromatic carbocyclyl. R^{X5} is preferably O, and R^{X6} is preferably a hydrogen atom.

The term “protective group used in nucleic acid synthesis” for R^{X4} and R^{X6} means a protective group of the above hydroxyl group. Preferable examples of the protective group include alkyl, alkenyl, “aliphatic acyl”, “aromatic acyl”, “methyl substituted by 1 to 3 aryl”, “aryl substituted by halogen, alkyloxy or nitro” and the like. More preferably, it is benzoyl, benzyl, 2-chlorophenyl, 4-chlorophenyl, 2-propenyl or the like.

The reactive phosphorus group is especially preferably diisopropylcyanoethoxy phosphoramidite (a group of the formula: —P(OC₂H₄CN)(N(i-Pr)₂)), H-phosphonate (a group of the formula: —P(═O)H(OH)) or the like.

The term “halogen” includes a fluorine atom, a chlorine atom, a bromine atom and an iodine atom. A fluorine atom and a chlorine atom are especially preferable.

The term “alkyl” includes a C1 to C15, preferably C1 to C10, more preferably C1 to C6 and further preferably C1 to C4 linear or branched hydrocarbon group. Examples include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl, n-decyl and the like.

A preferred embodiment of “alkyl” is methyl, ethyl, n-propyl, isopropyl, n-butyl, isobutyl, sec-butyl, tert-butyl or n-pentyl. A more preferred embodiment is methyl, ethyl, n-propyl, isopropyl or tert-butyl.

The term “alkenyl” includes a C2 to C15, preferably C2 to C10, more preferably C2 to C6 and further preferably C2 to C4 linear or branched hydrocarbon group having one or more double bond(s) at any position(s). Examples include vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl, undecenyl, dodecenyl, tridecenyl, tetradecenyl, pentadecenyl and the like.

A preferred embodiment of “alkenyl” is vinyl, allyl, propenyl, isopropenyl or butenyl.

The term “alkynyl” includes a C2 to C10, preferably a C2 to C8, more preferably a C2 to C6 and further preferably a C2 to C4 linear or branched hydrocarbon group having one or more triple bond(s) at any position(s). Examples include ethynyl, propynyl, butynyl, pentynyl, hexynyl, heptynyl, octynyl, nonynyl, decynyl and the like. Furthermore, it may have double bond(s) at any position(s).

A preferred embodiment of “alkynyl” is ethynyl, propynyl, butynyl or pentynyl.

The term “aromatic carbocyclyl (aryl)” means a cyclic aromatic hydrocarbon group which is monocyclic or polycyclic having two or more rings. Examples include phenyl, naphthyl, anthryl, phenanthryl and the like.

A preferred embodiment of “aromatic carbocyclyl (aryl)” is phenyl.

The term “non-aromatic carbocyclyl” means a cyclic saturated hydrocarbon group or cyclic unsaturated non-aromatic hydrocarbon group, which is monocyclic or polycyclic having two or more rings. Examples of the non-aromatic carbocyclyl, which is polycyclic having two or more rings, include a fused ring group wherein a non-aromatic carbocyclyl, which is monocyclic or polycyclic having two or more rings, is fused with a ring of the above “aromatic carbocyclyl”.

In addition, examples of the “non-aromatic carbocyclyl” also include a group having a bridge or a group to form a spiro ring as follows:

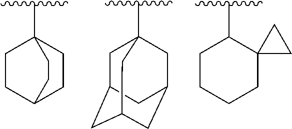

The non-aromatic carbocyclyl, which is monocyclic, is preferably C3 to C16, more preferably C3 to C12 and further preferably C4 to C8 carbocyclyl. Examples include cyclopropyl, cyclobutyl, cyclopentyl, cyclohexyl, cycloheptyl, cyclooctyl, cyclononyl, cyclodecyl, cyclopropenyl, cyclobutenyl, cyclopentenyl, cyclohexenyl, cycloheptenyl, cyclohexadienyl and the like.

Examples of non-aromatic carbocyclyl, which is polycyclic having two or more rings, include indanyl, indenyl, acenaphthyl, tetrahydronaphthyl, fluorenyl and the like.

The term “aromatic heterocyclyl” means an aromatic cyclyl, which is monocyclic or polycyclic having two or more rings, containing one or more of heteroatom(s) selected independently from O, S and N.

Examples of aromatic heterocyclyl, which is polycyclic having two or more rings, include a fused ring group wherein an aromatic heterocyclyl, which is monocyclic or polycyclic having two or more rings, is fused with a ring of the above “aromatic carbocyclyl”.

The aromatic heterocyclyl, which is monocyclic, is preferably a 5- to 8-membered and more preferably 5- to 6-membered ring. Examples include pyrrolyl, imidazolyl, pyrazolyl, pyridyl, pyridazinyl, pyrimidinyl, pyrazinyl, triazolyl, triazinyl, tetrazolyl, furyl, thienyl, isoxazolyl, oxazolyl., oxadiazolyl, isothiazolyl, thiazolyl, thiadiazolyl and the like.

Examples of aromatic heterocyclyl, which is bicyclic, include indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, oxazolopyridyl, thiazolopyridyl and the like.

Examples of aromatic heterocyclyl, which is polycyclic having three or more rings, include carbazolyl, acridinyl, xanthenyl, phenothiazinyl, phenoxathiinyl, phenoxazinyl, dibenzofuryl and the like.

The term “non-aromatic heterocyclyl” means a non-aromatic cyclyl, which is monocyclic or polycyclic having two or more rings, containing one or more heteroatom(s) selected independently from O, S and N.

Examples of non-aromatic heterocyclyl, which is polycyclic having two or more rings, include a fused ring group wherein a non-aromatic heterocycle, which is monocyclic or polycyclic having two or more rings, is fused with a ring of the above “aromatic carbocyclyl”, “non-aromatic carbocyclyl” and/or “aromatic heterocyclyl”.

In addition, examples of the “non-aromatic heterocyclyl” also include a group having a bridge or a group to form a spiro ring as follows:

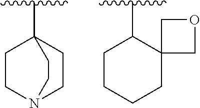

The non-aromatic heterocyclyl, which is monocyclic, is preferably a 3- to 8-membered and more preferably 5- to 6-membered ring. Examples include dioxanyl, thiiranyl, oxiranyl, oxetanyl, oxathiolanyl, azetidinyl, thianyl, thiazolidinyl, pyrrolidinyl, pyrrolinyl, imidazolidinyl, imidazolinyl, pyrazolidinyl, pyrazolinyl, piperidyl, piperazinyl, morpholinyl, morpholino, thiomorpholinyl, thiomorpholino, dihydropyridyl, tetrahydropyridyl, tetrahydrofuryl, tetrahydropyranyl, dihydrothiazolyl, tetrahydrothiazolyl, tetrahydroisothiazolyl, dihydrooxazinyl, hexahydroazepinyl, tetrahydrodiazepinyl, tetrahydropyridazinyl, hexahydropyrimidinyl, dioxolanyl, dioxazinyl, aziridinyl, dioxolinyl, oxepanyl, thiolanyl, thiinyl, thiazinyl and the like.

Examples of non-aromatic heterocyclyl, which is polycyclic having two or more rings, include indolinyl, isoindolinyl, chromanyl, isochromanyl and the like.

The term “alkyloxy” means a group wherein the above “alkyl” is bonded to an oxygen atom. Examples include methyloxy, ethyloxy, n-propyloxy, isopropyloxy, n-butyloxy, tert-butyloxy, isobutyloxy, sec-butyloxy, pentyloxy, isopentyloxy, hexyloxy and the like.

A preferred embodiment of “alkyloxy” is methyloxy, ethyloxy, n-propyloxy, isopropyloxy or tert-butyloxy.

The term “haloalkyl” means a group wherein one or more “halogen” described above is bonded to the above “alkyl”. Examples include monofluoromethyl, monofluoroethyl, monofluoropropyl, 2,2,3,3,3-pentafluoropropyl, monochloromethyl, trifluoromethyl, trichloromethyl, 2,2,2-trifluoroethyl, 2,2,2-trichloroethyl, 1,2-dibromoethyl, 1,1,1-trifluoropropan-2-yl and the like.

A preferred embodiment of “haloalkyl” is trifluoromethyl or trichloromethyl.

The term “alkylamino” includes monoalkylamino and dialkylamino.

The term “monoalkylamino” means a group wherein a hydrogen atom attached to a nitrogen atom of an amino group is replaced with the above “alkyl”. Examples include methylamino, ethylamino, isopropylamino and the like. Preferably, it is methylamino or ethylamino.

The term “dialkylamino” means a group wherein two hydrogen atoms attached to a nitrogen atom of an amino group are replaced with two “alkyl” described above. These two alkyl groups may be the same or different. Examples include dimethylamino, diethylamino, N,N-diisopropylamino, N-methyl-N-ethylamino, N-isopropyl-N-ethylamino and the like. Preferably, it is dimethylamino or diethylamino.

Examples of the substituents for “substituted or unsubstituted alkyl”, “substituted or unsubstituted alkenyl” and “substituted or unsubstituted alkynyl” include the following substituents. A carbon atom(s) at any position(s) may be bonded to one or more group(s) selected from the following substituents.

Substituents: halogen, hydroxy, carboxy, amino, imino, hydroxyamino, hydroxyimino, formyl, formyloxy, carbamoyl, sulfamoyl, sulfanyl, sulfino, sulfo, thioformyl, thiocarboxy, dithiocarboxy, thiocarbamoyl, cyano, nitro, nitroso, azide, hydrazino, ureide, amidino, guanidino, trialkylsilyl, alkyloxy, alkenyloxy, alkynyloxy, haloalkyloxy, alkylcarbonyl, alkenylcarbonyl, alkynylcarbonyl, monoalkylamino, dialkylamino, alkylsulfonyl, alkenylsulfonyl, alkynylsulfonyl, monoalkylcarbonylamino, dialkylcarbonylamino, monoalkylsulfonylamino, dialkylsulfonylamino, alkylimino, alkenylimino, alkynylimino, alkylcarbonyliniino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynyloxycarbonyl, alkylsulfanyl, alkenylsulfanyl, alkynylsulfanyl, alkylsulfinyl, alkenylsulfinyl, alkynylsulfinyl, monoalkylcarbamoyl, dialkylcarbamoyl, monoalkylsulfamoyl, dialkylsulfamoyl, aromatic carbocyclyl, non-aromatic carbocyclyl, aromatic heterocyclyl, non-aromatic heterocyclyl, aromatic carbocyclyloxy, non-aromatic carbocyclyloxy, aromatic heterocyclyloxy, non-aromatic heterocyclyloxy, aromatic carbocyclylcarbonyl, non-aromatic carbocyclylcarbonyl, aromatic heterocyclylcarbonyl, non-aromatic heterocyclylcarbonyl, aromatic carbocyclyloxycarbonyl; non-aromatic carbocyclyloxycarbonyl, aromatic heterocyclyloxycarbonyl, non-aromatic heterocyclyloxycarbonyl, aromatic carbocyclylalkyloxy, non-aromatic carbocyclylalkyloxy, aromatic heterocyclylalkyloxy, non-aromatic heterocyclylalkyloxy, aromatic carbocyclylalkyloxycarbonyl, non-aromatic carbocyclylalkyloxycarbonyl, aromatic heterocyclylalkyloxycarbonyl, non-aromatic heterocyclylalkyloxycarbonyl, aromatic carbocyclylalkylamino, non-aromatic carbocyclylalkylamino, aromatic heterocyclylalkylamino, non-aromatic heterocyclylalkylamino, aromatic carbocyclylsulfanyl, non-aromatic carbocyclylsulfanyl, aromatic heterocyclylsulfanyl, non-aromatic heterocyclylsulfanyl, non-aromatic carbocyclylsulfonyl, aromatic carbocyclylsulfonyl, aromatic heterocyclylsulfonyl and non-aromatic heterocyclylsulfonyl.

Examples of the substituents on the ring of “aromatic carbocycle” in “substituted or unsubstituted aromatic carbocyclyl” include the following substituents. An atom(s) at any position(s) on the ring may be bonded to one or more group(s) selected from the following substituents.

Substituents: halogen, hydroxy, carboxy, amino, imino, hydroxyamino, hydroxyimino, formyl, formyloxy, carbamoyl, sulfamoyl, sulfanyl, sulfino, sulfo, thioformyl, thiocarboxy, dithiocarboxy, thiocarbamoyl, cyano, nitro, nitroso, azide, hydrazino, ureide, amidino, guanidino, trialkylsilyl, alkyl, alkenyl, alkynyl, haloalkyl, alkyloxy, alkenyloxy, alkynyloxy, haloalkyloxy, alkyloxyalkyl, alkyloxyalkyloxy, alkylcarbonyl, alkenylcarbonyl, alkynylcarbonyl, monoalkylamino, dialkylamino, alkylsulfonyl, alkenylsulfonyl, alkynylsulfonyl, monoalkylcarbonylamino, dialkylcarbonylamino, monoalkylsulfonylamino, dialkylsulfonylamino, alkylimino, alkenylimino, alkynylimino, alkylcarbonylimino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynyloxycarbonyl, alkylsulfanyl, alkenylsulfanyl, alkynylsulfanyl, alkylsulfinyl, alkenylsulfinyl, alkynylsulfinyl, monoalkylcarbamoyl, dialkylcarbamoyl, monoalkylsulfamoyl, dialkylsulfamoyl, aromatic carbocyclyl, non-aromatic carbocyclyl, aromatic heterocyclyl, non-aromatic heterocyclyl, aromatic carbocyclyloxy, non-aromatic carbocyclyloxy, aromatic heterocyclyloxy, non-aromatic heterocyclyloxy, aromatic carbocyclylcarbonyl, non-aromatic carbocyclylcarbonyl, aromatic heterocyclylcarbonyl, non-aromatic heterocyclylcarbonyl, aromatic carbocyclyloxycarbonyl, non-aromatic carbocyclyloxycarbonyl, aromatic heterocyclyloxycarbonyl, non-aromatic heterocyclyloxycarbonyl, aromatic carbocyclylalkyl, non-aromatic carbocyclylalkyl, aromatic heterocyclylalkyl, non-aromatic heterocyclylalkyl, aromatic carbocyclylalkyloxy, non-aromatic carbocyclylalkyloxy, aromatic heterocyclylalkyloxy, non-aromatic heterocyclylalkyloxy, aromatic carbocyclylalkyloxycarbonyl, non-aromatic carbocyclylalkyloxycarbonyl, aromatic heterocyclylalkyloxycarbonyl, non-aromatic heterocyclylalkyloxycarbonyl, aromatic carbocyclylalkyloxyalkyl, non-aromatic carbocyclylalkyloxyalkyl, aromatic heterocyclylalkyloxyalkyl, non-aromatic heterocyclylalkyloxyalkyl, aromatic carbocyclylalkylamino, non-aromatic carbocyclylalkylamino, aromatic heterocyclylalkylamino, non-aromatic heterocyclylalkylamino, aromatic carbocyclylsulfanyl, non-aromatic carbocyclylsulfanyl, aromatic heterocyclylsulfanyl, non-aromatic heterocyclylsulfanyl, non-aromatic carbocyclylsulfonyl, aromatic carbocyclylsulfonyl, aromatic heterocyclylsulfonyl and non-aromatic heterocyclylsulfonyl.

Preferred embodiments of a compound of formula (I) of the present invention are disclosed below.

Y¹-Y² is S(═O)—NR⁶, S(═O)₂—NR⁶, NR⁶—S(═O) or NR⁶—S(═O)₂. Preferably, it is S(═O)₂—NR⁶ or NR⁶—S(═O)₂. More preferably, it is S(═O)₂—NR⁶.

R⁶ is a hydrogen atom, substituted or unsubstituted alkyl, substituted or unsubstituted alkenyl, or substituted or unsubstituted alkynyl. Preferably, it is a hydrogen atom, or substituted or unsubstituted alkyl. More preferably, it is a hydrogen atom or alkyl.

Bx is a nucleic acid base moiety. More preferably, it is substituted or unsubstituted purin-9-yl, or substituted or unsubstituted 2-oxo-pyrimidin-1-yl.

Z¹ is each independently, a hydrogen atom, a hydroxyl protecting group or a reactive phosphorus group. Preferably, it is a hydrogen atom or a hydroxyl protecting group. More preferably, it is a hydrogen atom, acetyl, t-butyl, t-butoxymethyl, methoxymethyl, tetrahydropyranyl, 1-ethoxyethyl, 1-(2-chloroethoxy)-ethyl, 2-trimethylsilylethyl, p-chlorophenyl, 2,4-dinitrophenyl, benzyl, benzoyl, p-phenylbenzoyl, 2,6-dichlorobenzyl, levulinoyl, diphenylmethyl, p-nitrobenzyl, trimethylsilyl, triethylsilyl, t-butyldimethylsilyl, t-butyldiphenylsilyl, triphenylsilyl, triisopropylsilyl, benzoyl formate, chloroacetyl, trichloroacetyl, trifluoroacetyl, pivaloyl, isobutyryl, 9-fluorenylmethyloxycarbonyl, methansulfonyl, p-toluenesulfonyl, trifluoromethanesulfonyl, triphenylmethyl (trityl), monomethoxytrityl, dimethoxytrityl (DMTr), trimethoxytrityl, 9-phenylxanthin-9-yl (Pixyl) or 9-(p-methoxyphenyl) xanthin-9-yl (MOX). Especially preferably, it is a hydrogen atom, benzyl, triethylsilyl, t-butyldimethylsilyl, t-butyldiphenylsilyl, triisopropylsilyl, trityl, monomethoxytrityl, dimethoxytrityl, trimethoxytrityl or the like.

Z² is each independently, a hydrogen atom, a hydroxyl protecting group or a reactive phosphorus group. Preferably, it is a hydrogen atom or a reactive phosphorus group. More preferably, it is a hydrogen atom, diisopropylcyanoethoxy phosphoramidite or H-phosphonate.

R¹ and R² are each independently, a hydrogen atom, halogen, cyano, substituted or unsubstituted alkyl, substituted or unsubstituted alkenyl, or substituted or unsubstituted alkynyl. Preferably, it is a hydrogen atom or alkyl.

R³ are each independently, a hydrogen atom, halogen, cyano, substituted or unsubstituted alkyl, substituted or unsubstituted alkenyl, or substituted or unsubstituted alkynyl. Preferably, it is a hydrogen atom, halogen, cyano or alkyl.

R⁴ are each independently, a hydrogen atom, halogen, cyano, substituted or unsubstituted alkyl, substituted or unsubstituted alkenyl, or substituted or unsubstituted alkynyl. Preferably, it is a hydrogen atom, halogen, cyano or alkyl.

R⁵ is a hydrogen atom, halogen, cyano, substituted or unsubstituted alkyl, substituted or unsubstituted alkenyl, or substituted or unsubstituted alkynyl. Preferably, it is a hydrogen atom, halogen, cyano, or alkyl.

n is an integer of 0 to 3. Preferably, it is 0 or 1.

The compounds of formula (I) are not limited to specific isomers but include all possible isomers (e.g., keto-enol isomers, imine-enamine isomers, diastereoisomers, enantiomers, rotamers or the like), racemates or mixtures thereof.

One or more hydrogen, carbon and/or other atoms in the compounds of formula (I) may be replaced with isotopes of hydrogen, carbon and/or other atoms respectively. Examples of the isotopes include hydrogen, carbon, nitrogen, oxygen, phosphorus, sulfur, fluorine, iodine and chlorine, such as ²H, ³H, ¹¹C, ¹³C, ¹⁴C, ¹⁵N, ¹⁸O, ¹⁷O, ³¹P, ³²P, ³⁵S, ¹⁸F, ¹²³I and ³⁶Cl respectively. The compounds of formula (I) include compounds replaced with these isotopes. The compounds replaced with the above isotopes are useful as pharmaceuticals and include all of radiolabeled compounds of the compound of formula (I). A “method of radiolabeling” in the manufacture of the “radiolabeled compounds” is encompassed by the present invention, and the “radiolabeled compounds” are useful for studies on metabolized drug pharmacokinetics, studies on binding assay and/or diagnostic tools.

A radiolabeled compound of formula (I) can be prepared using well-known methods in this field of the invention. For example, a tritium-labeled compound of formula (I) can be prepared by introducing a tritium to a compound of formula (I), through a catalytic dehalogenation reaction using a tritium. This method comprises reacting with an appropriately-halogenated precursor of the compound of formula (I) with tritium gas in the presence of an appropriate catalyst, such as Pd/C, and in the presence or absent of a base. The other appropriate method of preparing a tritium-labeled compound can be referred to “Isotopes in the Physical and Biomedical Sciences, Vol. 1, Labeled Compounds (Part A), Chapter 6 (1987)”. A ¹⁴C-labeled compound can be prepared by using a raw material having ¹⁴C carbon.

The present invention encompasses preparable salts of the compounds of formula (I). The salts include, for example, alkaline metal salts such as sodium salts, potassium salts, lithium salts and the like; alkaline earth metal salts such as calcium salts, magnesium salts and the like; metal salts such as aluminum salts, iron salts, zinc salts, copper salts, nickel salts, cobalt salts and the like; ammonium salt; amine salts such as t-octylamine salts, dibenzylamine salts, morpholine salts, glucosamine salts, phenylglycine alkylester salts, ethylenediamine salts, N-methylglucamine salts, guanidine salts, diethylamine salts, triethylamine salts, dicyclohexylamine salts, N,N′-dibenzylethylenediamine salts, claloroprocaine salts, procaine salts, diethanolamine salts, N-benzyl-phenethylamine salts, piperazine salts, tetramethylammonium salts, Tris(hydroxymethyl)aminomethane salts and the like; inorganic acid salts such as halide acid salts (hydrofluoride, hydrochloride, hydrobromide, hydriodide and the like), nitrates, perchlorates, sulfates, phosphates and the like; alkanesulfonates such as methanesulfonates, trifluoromethanesulfonates, ethanesulfonates and the like; arylsulfonates salts such as benzenesulfonates, p-toluenesulfonates and the like; organic acid salts such as acetates, malates, fumarates, succinates, citrates, tartrates, oxalates, maleates and the like; amino acid salts such as glycine salts, lysine salts, arginine salts, ornithine salts, glutamates, aspartates and the like; etc. These salts can be formed by the usual methods.

The compounds of formula (I) of the present invention or salts thereof may form solvates (e.g., hydrates or the like) and/or crystal polymorphs. The present invention encompasses those various solvates and crystal polymorphs. “Solvates” may be those wherein any numbers of solvent molecules (e.g., water molecules or the like) are coordinated with the compounds of formula (I). When the compounds of formula (I) or pharmaceutically acceptable salts thereof are allowed to stand in the atmosphere, the compounds may absorb water, resulting in attachment of adsorbed water or formation of hydrates. Recrystallization of the compounds of formula (I) or pharmaceutically acceptable salts thereof may produce crystal polymorphs.

Compounds of formula (I) of the present invention can be synthesized based on the publicly known methods in this field. For example, they can be produced by the general synthetic methods described below. Additionally, the methods for extraction, purification and the like may be carried out by using the usual methods for the experiments of organic chemistry.

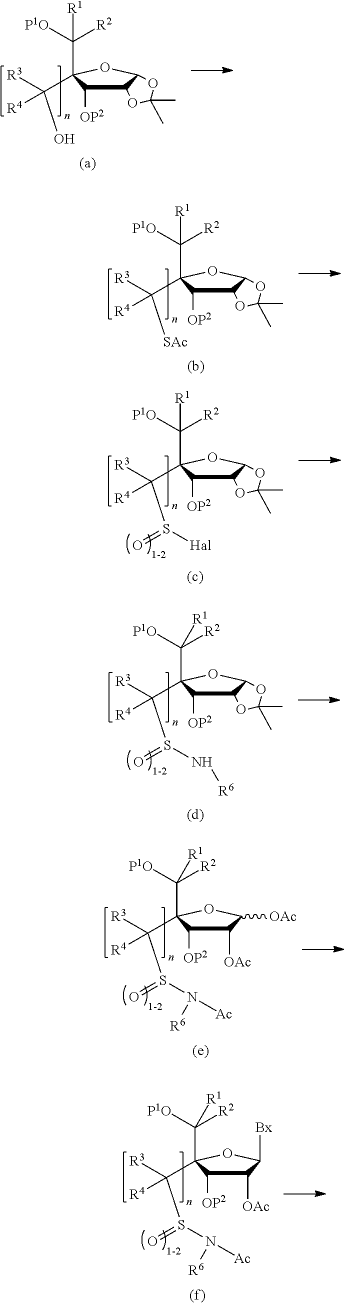

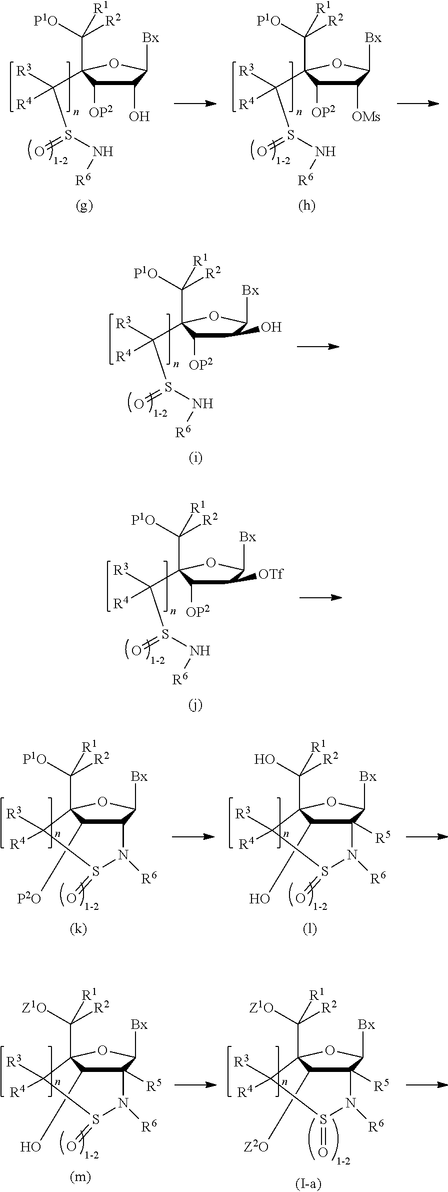

wherein P¹ and P² are each independently, a hydroxyl protecting group, preferably benzyl, naphthyl, t-butyldimethylsilyl, t-butyldiphenylsilyl or benzoyl. Z¹ is a hydroxyl protecting group, preferably t-butyldimethylsilyi, t-butyldiphenylsilyl, triisopropylsilyl, trityl, monomethoxytrityl, dimethoxytrityl or trimethoxytrityl. Z² is a reactive phosphorus group, preferably diisopropylcyanoethoxy phosphoramidite or H-phosphonate. Each of the other symbols has the same meaning as those of a compound of the formula (I).

A hydroxyl group of compound (a) is converted into a thioacetyl group to give compound (b). The thioacetyl group is converted into sulfonyl chloride or sulfinyl chloride to give compound (c). Next, after reacting with substituted amine to give compound (d), deprotection of acetonide and protection of a hydroxyl group and a sulfonamide group are carried out together to give compound (e). After introducing a base moiety to give compound (f), the acetyl group is removed to give compound (g). A hydroxyl group at 2′-position of compound (g) is converted into a mesyl group to give compound (h). After reacting compound (h) with sodium acetate, sodium benzoate or the like, hydrolysis is carried out to give compound (i). After converting into compound (j) by changing the hydroxyl group at 2′-position to triflate, it is treated with base to give compound (k). Subsequently, the protective groups of the hydroxyl groups at 3′-position and 5′-position are removed, and a substituent is introduced into R⁵ as needed, to give compound (l). A protective group (especially, a trityl group optionally substituted by a methoxy group) is introduced into the hydroxyl group at 5′-position to give compound (m). A reactive phosphorus group (especially, diisopropylcyanoethoxy phosphoramidite) is introduced into the hydroxyl group at 3′-position to give compound (I-a).

Alternatively, for example, as Example 1 of this description, when a compound wherein Bx is thymine, and P¹ and P² are benzyl (compound k-1) was obtained by the above method, it can be converted into the other nucleic acid base by the following method.

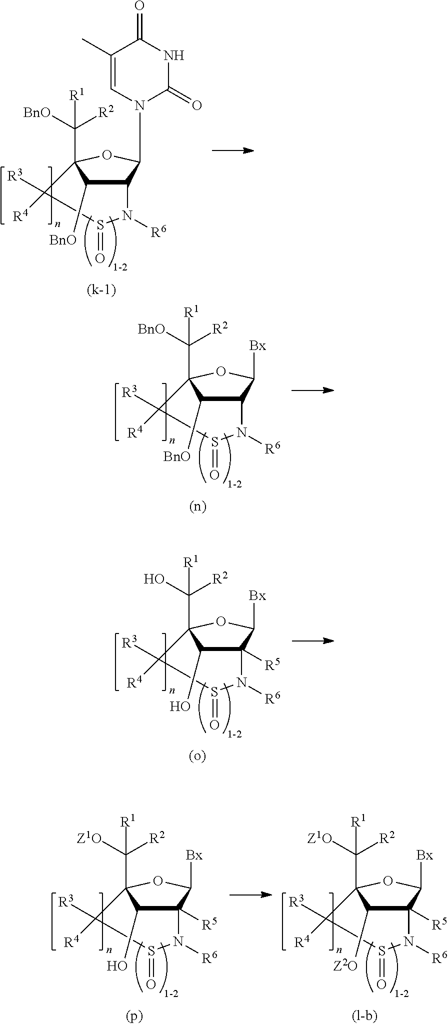

wherein Bx is a nucleic acid base except for thymine, preferably guanine or adenine. Z¹ is a hydroxyl protecting group, preferably t-butyldimethylsilyl, t-butyldiphenylsilyl, triisopropylsilyl, trityl, monomethoxytrityl, dimethoxytrityl or trimethoxytrityl. Z² is a reactive phosphorus group, preferably diisopropylcyanoethoxy phosphoramidite or H-phosphonate. Each of the other symbols has the same meaning as those of a compound of the formula (I).

### Step 1 Synthesis of Compound (n)

Compound (k−1) obtained by the above method are reacted with a silylated nucleic acid base moiety in the solvent such as acetonitrile, 1,2-dichloroethane, toluene or the like, or in a mixed solvent thereof, under the presence of acid such as trimethylsilyl trifluoromethanesulfonate or tin tetrachloride or the like, at 25° C. to 140° C. for 1 to 24 hours to give compound (n).

### Step 2 Synthesis of Compound (o)

To compound (n) in the solvent such as methanol, ethanol, tetrahydrofuran or the like, or in a mixed solvent thereof is added palladium-carbon powder or palladium hydroxide-carbon powder, and the mixture is reacted under hydrogen stream at 0° C. to 40° C. for 1 to 24 hours to give compound (o). A substituent can be introduced into R⁵ as needed.

### Step 3 Synthesis of Compound (p)

To compound (o) in pyridine is added 4,4′-dimethoxytrityl chloride or the like, and the mixture is reacted at 0° C. to 80° C. for 1 to 24 hours to give compound (p).

### Step 4 Synthesis of Compound (I-b)

To compound (p) in the solvent such as acetonitrile, dichloromethane, tetrahydrofuran or the like, or in a mixed solvent thereof, is added 2-cyanoethyl-N,N-diisopropylchloro phosphoramidite or the like under the presence of base such as diisopropylethylamine, triethylamine or the like, or 2-cyanoethyl-N,N,N′,N′-tetraisopropyl phosphoramidite or the like under the presence of acid such as tetrazole, dicyanoimidazole or the like, and the mixture is reacted at 0° C. to 60° C. for 1 to 24 hours to give compound (I-b).

In the above steps, the following intermediates are useful.

A compound of the formula:

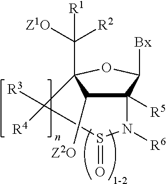

wherein Z¹ and Z² are each independently, a hydrogen atom or a hydroxyl protecting group. Each of the other symbols has the same meaning as those of a compound of the formula (I).

Z¹ is preferably hydrogen, benzyl, t-butyldimethylsilyl, t-butyldiphenylsilyl, triisopropylsilyl, trityl, monomethoxytrityl, dimethoxytrityl or trimethoxytrityl. Especially preferably, it is hydrogen, benzyl or dimethoxytrityl.

Z² is preferably hydrogen, benzyl or triethylsilyl.

The nucleoside of the present invention means a compound of compound (I) wherein Z¹ and Z² is hydrogen.

The nucleotide of the present invention means a compound of compound (I) wherein Z² is a reactive phosphorus group.

The present invention encompasses a following oligonucleotide prepared with a compound of formula (I), or a pharmaceutically acceptable salt thereof.

An oligonucleotide comprising one or more nucleoside structure of formula (II) or a pharmaceutically acceptable salt thereof:

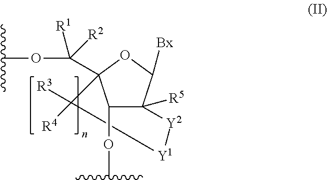

wherein Y¹-Y² is S(═O)—NR⁶, S(═O)₂—NR⁶, NR⁶—S(═O) or NR⁶—S(═O)₂,
R⁶ is a hydrogen atom, substituted or unsubstituted alkyl, substituted or unsubstituted alkenyl, or substituted or unsubstituted alkynyl,
Bx is a nucleic acid base moiety,
R¹ and R² are each independently, a hydrogen atom, halogen, cyano, substituted or unsubstituted alkyl, substituted or unsubstituted alkenyl, or substituted or unsubstituted alkynyl,
R³ are each independently, a hydrogen atom, halogen, cyano, substituted or unsubstituted alkyl, substituted or unsubstituted alkenyl, or substituted or unsubstituted alkynyl,
R⁴ are each independently, a hydrogen atom, halogen, cyano, substituted or unsubstituted alkyl, substituted or unsubstituted alkenyl, or substituted or unsubstituted alkynyl,
R⁵ is a hydrogen atom, halogen, cyano, substituted or unsubstituted alkyl, substituted or unsubstituted alkenyl, or substituted or unsubstituted alkynyl, and
n is an integer of 0 to 3.

Each of the symbols of the formula has the same meaning as those of a compound of the formula (I).

The oligonucleotide of the present invention is an oligonucleotide whose length is 2 to 50 bases, and preferably 8 to 30 bases, and which comprises at least one nucleoside structure of formula (II) at any position(s). The position and number of the nucleoside structures are not limited to the specific position and number and may be appropriately selected depending on the purposes. For example, the nucleoside structure of formula (II) can be comprised at the 3′-terminus or 5′-terminus of the oligonucleotide. When it is comprised at the 3′-terminus, the structure is as below.

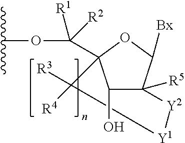

wherein each of the symbols has the same meaning as those of a compound of the formula (I).

When it is comprised at the 5′-terminus, the structure is as below.

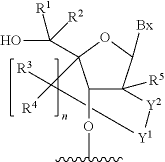

wherein each of the symbols has the same meaning as those of a compound of the formula (I).

The 3′-terminus and/or 5′-terminus of an oligonucleotide of the present invention can be modified. The modified groups publicly known in this field can be used to allow tracking of the oligonucleotide, to improve pharmacokinetics or pharmacodynamics of the oligonucleotide, or to enhance stability or binding affinity of the oligonucleotide. Examples include protective groups for a hydroxyl group, reporter molecule, cholesterol, phospholipid, dye, fluorescent molecule and the like.

Alternatively, the 3′-terminus and/or 5′-terminus of an oligonucleotide of the present invention can comprise phosphate ester moieties. The term “phosphate ester moiety” means a phosphate group at the terminus comprising phosphoester or modified phosphoester. The phosphate ester moiety can be located at either terminus, but preferably it is a 5′-terminus nucleoside. Concretely, it is a group of the formula: —O—P(═O)(OH)OH or a modified group thereof. In other words, one or more O or OH is optionally substituted by H, O, S, N(R^{X}), or alkyl wherein R^{X} is H, an amino protecting group, or substituted or unsubstituted alkyl. A group at the 5′- and/or 3′-terminus can comprise each independently substituted or unsubstituted 1 to 3 phosphate ester moiety.

As long as an oligonucleotide of the present invention comprise at least one nucleoside structure of formula (II), the other parts can be same with natural nucleic acids or have nucleotide modifications publicly known in this field.

Examples of a phosphate moiety of an oligonucleotide of the present invention include phosphodiester linkage comprised in natural nucleic acids, S-oligo (phosphorothioate), M-oligo (methylphosphonate), borano phosphate and the like.

A base moiety except for nucleoside structures of formula (II) in an oligonucleotide of the present invention can be any nucleic acid base defined as the above “Bx”.

Examples of a sugar moiety except for nucleoside structures of formula (II) in an oligonucleotide of the present invention are natural ribose or deoxyribose, ribose or deoxyribose with the publicly known modification, and the like. Examples of the publicly known modifications are modifications by 2′-O—CH₂—CH₂—O—CH₃ (2′MOE), 4′-CH₂—O—2′ (LNA, Locked nucleic acid), AmNA (amideBNA) (Bridged nucleic acid, WO2011/052436) and the like.

Additionally, internucleoside linkages comprised in an oligonucleotide of the present invention can be linkages not having a phosphorus atom as long as they are publicly known in this field. They include alkyl, non-aromatic carbocycle, non-aromatic carbocycle substituted by haloalkyl or halogen and the like, but are not limited to them. Examples include siloxane, sulfide, sulfoxide, sulfone, acetyl, acetyl formate, thioacetyl formate, methyleneacetyl formate, thioacetyl formate, alkenyl, sulfamate, methyleneimino, methylenehydrazino, sulfonate, sulfonamide and amide.

The oligonucleotides of the present invention are not limited to specific isomers but include all possible isomers (e.g., keto-enol isomers, imine-enamine isomers, diastereoisomers, enantiomers, rotamers or the like), racemates or mixtures thereof.

One or more hydrogen, carbon and/or other atoms in the oligonucleotides of the present invention may be replaced with isotopes of hydrogen, carbon and/or other atoms respectively. Examples of the isotopes include hydrogen, carbon, nitrogen, oxygen, phosphorus, sulfur, fluorine, iodine and chlorine, such as ²H, ³H, ¹¹C, ¹³C, ¹⁴C, ¹⁵N, ¹⁸O, ¹⁷O, ³¹P, ³²P, ³⁵S, ¹⁸F, ¹²³I and ³⁶Cl respectively. The oligonucleotides of the present invention include the compounds replaced with these isotopes. The compounds replaced with the above isotopes are useful as pharmaceuticals and include all of radiolabeled compounds of the oligonucleotides of the present invention. A “method of radiolabeling” in the manufacture of the “radiolabeled compounds” is encompassed by the present invention, and the “radiolabeled compounds” are useful for studies on metabolized drug pharmacokinetics, studies on binding assay and/or diagnostic tools.

A radiolabeled compound of the oligonucleotides of the present invention can be prepared using well-known methods in this field of the invention. For example, a tritium-labeled oligonucleotide of the present invention can be prepared by introducing a tritium to an oligonucleotide of the present invention, through a catalytic dehalogenation reaction using a tritium. This method comprises reacting with an appropriately-halogenated precursor of the oligonucleotide of the present invention with tritium gas in the presence of an appropriate catalyst, such as Pd/C, and in the presence or absent of a base. The other appropriate method of preparing a tritium-labeled compound can be referred to “Isotopes in the Physical and Biomedical Sciences, Vol. 1, Labeled Compounds (Part A), Chapter 6 (1987)”. A ¹⁴C-labeled compound can be prepared by using a raw material having ¹⁴C carbon.

The present invention encompasses pharmaceutically acceptable salts of the oligonucleotides of the present invention. The salts include, for example, alkaline metal salts such as sodium salts, potassium salts, lithium salts and the like; alkaline earth metal salts such as calcium salts, magnesium salts and the like; metal salts such as aluminum salts, iron salts, zinc salts, copper salts, nickel salts, cobalt salts and the like; ammonium salt; amine salts such as t-octylamine salts, dibenzylamine salts, morpholine salts, glucosamine salts, phenylglycine alkylester salts, ethylenediamine salts, N-methylglucamine salts, guanidine salts, diethylamine salts, triethylamine salts, dicyclohexylamine salts, N,N′-dibenzylethylenediamine salts, chloroprocaine salts, procaine salts, diethanolamine salts, N-benzyl-phenethylamine salts, piperazine salts, tetramethylammonium salts, Tris(hydroxymethyl)aminomethane salts and the like; inorganic acid salts such as halide acid salts (hydrofluoride, hydrochloride, hydrobromide, hydriodide and the like), nitrates, perchlorates, sulfates, phosphates and the like; alkanesulfonates such as methanesulfonates, trifluoromethanesulfonates and ethanesulfonates; arylsulfonates salts such as benzenesulfonates, p-toluenesulfonates and the like; organic acid salts such as acetates, malates, fumarates, succinates, citrates, tartrates, oxalates, maleates and the like; amino acid salts such as glycine salts, lysine salts, arginine salts, ornithine salts, glutamates, aspartates and the like; etc. These salts can be formed by the usual methods.

The oligonucleotides of the present invention or pharmaceutically acceptable salts thereof may form solvates (e.g., hydrates or the like) and/or crystal polymorphs. The present invention encompasses those various solvates and crystal polymorphs. “Solvates” may be those wherein any numbers of solvent molecules (e.g., water molecules or the like) are coordinated with the oligonucleotides of the present invention. When the oligonucleotides of the present invention or pharmaceutically acceptable salts thereof are allowed to stand in the atmosphere, the compounds may absorb water, resulting in attachment of adsorbed water or formation of hydrates. Recrystallization of the oligonucleotides of the present invention or pharmaceutically acceptable salts thereof may produce crystal polymorphs.

The oligonucleotides or pharmaceutically acceptable salts thereof of the present invention may form prodrugs. The present invention also encompasses such various prodrugs. Prodrugs are derivatives of the compounds of the present invention with a chemically or metabolically degradable group(s), and compounds that are converted to the pharmaceutically active oligonucleotide of the present invention through solvolysis or under physiological conditions in vivo. Prodrugs include compounds that are converted to the oligonucleotides of the present invention through enzymatic oxidation, reduction, hydrolysis or the like under physiological conditions in vivo, compounds that are converted to the oligonucleotides of the present invention through hydrolysis by gastric acid etc., and the like. Methods for selecting and preparing suitable prodrug derivatives are described in, for example, “Design of Prodrugs, Elsevier, Amsrdam, 1985”. Prodrugs themselves may have some activity.

When the oligonucleotides or pharmaceutically acceptable salts thereof of the present invention have hydroxyl group(s), the prodrugs include acyloxy derivatives and sulfonyloxy derivatives that are prepared by, for example, reacting compounds having hydroxyl group(s) with suitable acyl halide, suitable acid anhydride, suitable sulfonyl chloride, suitable sulfonyl anhydride or mixed anhydride, or with a condensing agent. Examples include CH₃COO—, C₂H₅COO—, tert-BuCOO—, C₁₅H₃₁COO—, PhCOO—, (m-NaOOCPh)COO—, NaOOCCH₂CH₂COO—, CH₃CH(NH₂)COO—, CH₂N(CH₃COO—, CH₃SO₃—, CH₃CH₂SO₃—, CF₃SO₃—, CH₂FSO₃CF₃CH₂SO₃—, p-CH₃O-PhSO₃PhSO₃— and p-CHsPhSO₃.

The oligonucleotides of the present invention can be synthesized according to the usual methods with a compound of formula (I). For example, they can be easily synthesized by an automated nucleic acid synthesizer which is commercially available (e.g., the synthesizer by Applied Biosystems, Dainippon Seiki or the like). A method for synthesizing is solid-phase synthesis using phosphoramidite, solid-phase synthesis using hydrogen phosphonate or the like. For example, it disclosed in Tetrahedron Letters 22, 1859-1862 (1981), WO2011/052436 or the like.

Bx in a nucleoside structure of formula (II) is preferably that its substituent is not protected with a protective group. Examples include the following groups.

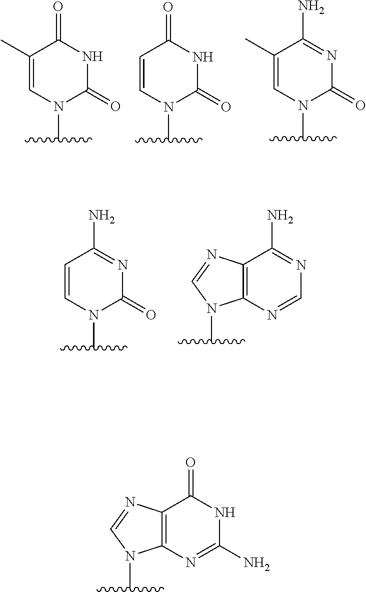

Therefore, when Bx in a compound of formula (I) has a substituent protected with a protective group, deprotection is carried out during the oligonucleotide synthesis.

The oligonucleotides of the present invention show the superior binding affinity to a single strand RNA and nuclease resistance. Therefore, the oligonucleotides are thought to have very good in vivo persistence. Then, compounds (I) of the present invention are useful very much as materials for synthesizing nucleic acid pharmaceuticals such as antisense oligonucleotide and the like. The nucleic acid pharmaceuticals using the oligonucleotides of the present invention have the high affinity to the target molecule compared to unmodified nucleic acid pharmaceuticals, are difficult to degrade in vivo, and then show more stable effects.

A nucleic acid pharmaceutical using the oligonucleotides of the present invention may be administered in a number of ways depending upon whether local or systemic treatment is desired and upon the area to be treated. As an administration method, for example, it may be topical (including ophthalmic, intravaginal, intrarectal, intranasal and transdermal), oral or parenteral. Parenteral administration includes intravenous injection or drip infusion, subdermal, intraperitoneal or intramuscular injection, lung administration by aspiration or inhalation, intrathecal administration, intraventricular administration and the like.

When a nucleic acid pharmaceutical using the oligonucleotides of the present invention is topically administered, a formulation such as a transdermal patche, ointment, lotion, cream, gel, drop, suppository, spray, liquid, powder or the like can be used.

The composition for oral administration includes powder, granule, suspension or solution dissolved in water or non-aqueous vehicle, capsule, powder, tablet or the like.

The composition for parenteral, intrathecal or intraventricular administration includes sterile aqueous solutions which contain buffers, diluents and other suitable additives, or the like.

A nucleic acid pharmaceutical using oligonucleotides of the present invention may be manufactured by mixing an effective amount of a nucleic acid with various pharmaceutical additives suitable for the administration form, such as excipients, binders, moistening agents, disintegrants, lubricants, diluents and the like. When it is an injection, an active ingredient together with a suitable carrier can be sterilized to give a pharmaceutical composition.

Examples of the excipients include lactose, saccharose, glucose, starch, calcium carbonate, crystalline cellulose and the like. Examples of the binders include methylcellulose, carboxymethylcellulose, hydroxypropylcellulose, gelatin, polyvinylpyrrolidone and the like. Examples of the disintegrants include carboxymethylcellulose, sodium carboxymethylcellulose, starch, sodium alginate, powdered agar, sodium lauryl sulfate and the like. Examples of the lubricants include talc, magnesium stearate, macrogol and the like. Cacao oil, macrogol, methylcellulose or the like may be used as base materials of suppositories. When the composition is manufactured as solutions, emulsified injections or suspended injections, solubilizing agent, suspending agents, emulsifiers, stabilizers, preservatives, isotonic agents and the like which are usually used may be added. For oral administration, sweetening agents, flavors and the like which are usually used may be added.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in vivo. Persons of ordinary skill in the art can easily determine optimal dosages, dosing methodologies and repetition rates. Optimal dosages can be generally calculated based on IC50 or EC50 in vitro or in vivo animal experiments although they change according to relative efficacy of each nucleic acid pharmaceutical. Dosages shown as mg/kg are calculated according to the usual method when, for example, a molecular weight of a nucleic acid (derived from the nucleic acid sequence and chemical structure) and effective dosage such as IC50 (derived from experiments) are provided.

In this description, meaning of each abbreviation is as follows:

Ac: acetyl
Bn: benzyl
DMTr: dimethoxytrityl
Et: ethyl
Hal: halogen
i-Pr: isopropyl
Me: methyl
Ms: methanesulfonyl
Nap: naphthylmethyl
Pac: phenoxyacetyl
Ph: phenyl
t-Bu: tert-butyl
TES: triethylsilyl
Tf: trifluoromethanesulfonyl

### EXAMPLES

The present invention is further explained by the following Examples, Reference Examples and Experiment Examples which are not intended to limit the scope of the present invention.

NMR analysis of compounds obtained in the examples was performed by 162 MHz, 300 MHz or 400 MHz using CD₃OD or CDCl₃.

### Example 1-1

### Synthesis of a Nucleotide of the Present Invention (Compound I-1)

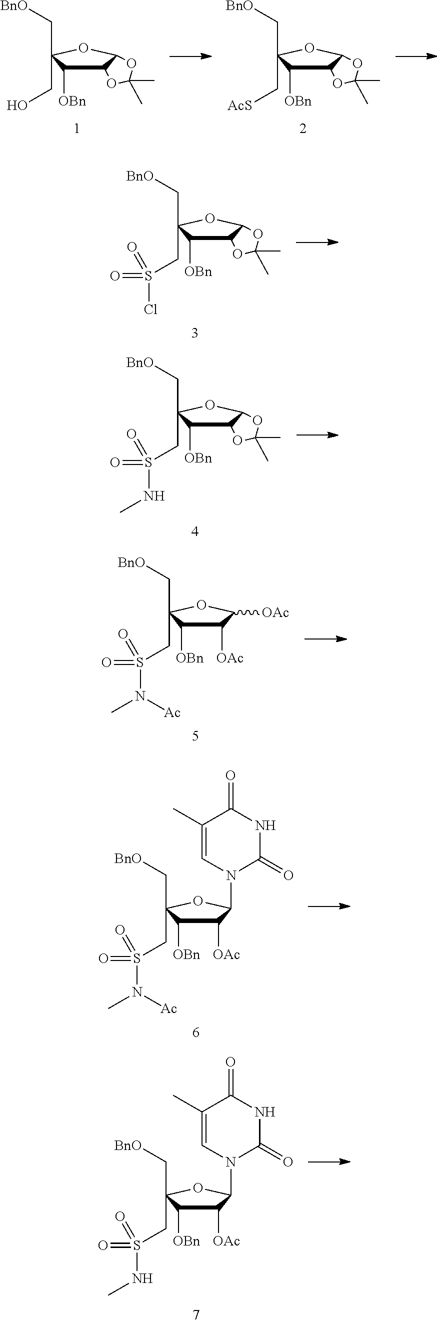

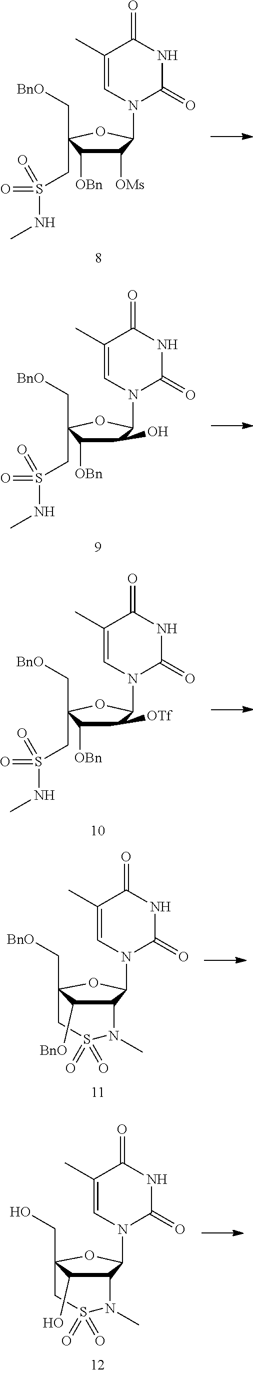

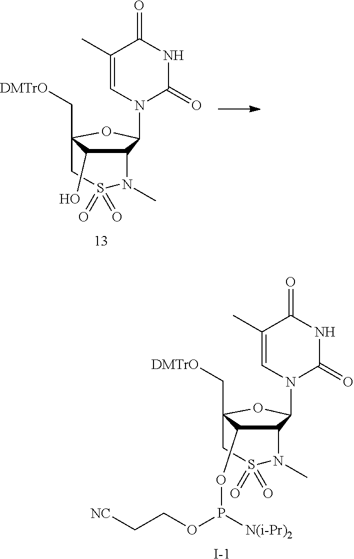

### Step 1 Synthesis of Compound 2

Under nitrogen stream, to a solution of triphenylphosphine (508 mg, 1.94 mmol) in tetrahydrofuran (6.5 mL) was added diisopropyl azodicarboxylate (376 μL, 1.94 mmol) on ice-cooling, and the mixture was stirred for 25 minutes. On ice-cooling, to the reaction solution were added a solution of compound 1 (J. Am. Chem. Soc. 2008, 130, 4886) (646 mg, 1.61 mmol) and thioacetic acid (139 μL, 1.94 mmol) in tetrahydrofuran (3.25 mL), and the mixture was stirred at room temperature for 4.5 hours. After the solvent was evaporated under reduced pressure, the resultant crude product was purified by silica gel column chromatography (n-hexane:ethyl acetate=90:10→70:30) to give compound 2 (682 mg, 92%) as colorless oil.

¹H-NMR (CDCl₃) δ: 1.32 (3H, s), 1.58 (3H, s), 2.32 (3H, s), 3.36-3.40 (2H, m), 3.46 (1H, d, J=10.5 Hz), 3.68 (1H, d, J=14.3 Hz), 4.23 (1H, d, J=5.1 Hz), 4.39 (1H, d, J=11.9 Hz), 4.49 (1H, d, J=11.9 Hz), 4.56-4.62 (2H, m), 4.76 (1H, d, J=11.9 Hz), 5.72 (1H, d, J=3.8 Hz), 7.22-7.37 (10H, m).

### Step 2 Synthesis of Compound 3

At room temperature, to a mixed solution of 2 mol/L hydrochloric acid solution (0.36 ml) and acetonitrile (1.8 ml) was added N-chlorosuccinimide (533 mg, 3.99 mmol), and the mixture was on ice-cooling. To the reaction solution was added a solution of compound 2 (457 mg, 0.997 mmol) in acetonitrile (1.2 ml), and the mixture was on ice-cooling and stirred for 30 minutes. To the reaction solution was added water, and the mixture was extracted with ethyl acetate. The organic layer was washed with brine, and then dried over sodium sulfate. The solvent was evaporated under reduced pressure, and the resultant crude product was purified by silica gel column chromatography (n-hexane:ethyl acetate=90:10→70:30) to give compound 3 (372 mg, 77%) as white solid.

¹H-NMR (CDCl₃) δ: 1.34 (3H, s), 1.70 (3H, s), 3.73 (1H, d, J=10.4 Hz), 3.79 (1H, d, J=10.4 Hz), 4.21-4.27 (2H, m), 4.54-4.60 (4H, m), 4.74 (1H, d, J=11.9 Hz), 4.80 (1H, d, J=14.8 Hz), 5.77 (1H, d, J=3.6 Hz), 7.26-7.37 (13H, m).

### Step 3 Synthesis of Compound 4

On ice-cooling, to 40% methylamine aqueous solution (0.85 mL) was added dropwise a solution of compound 3 (167 mg, 0.347 mmol) in tetrahydrofuran (1.7 mL), and the mixture was stirred for 30 minutes. After the solvent was evaporated under reduced pressure, to the residue was added water, and the mixture was extracted with ethyl acetate. The organic layer was washed with water and brine, and then dried over sodium sulfate. The solvent was evaporated under reduced pressure to give the crude product of compound 4 (156 mg).

¹H-NMR (CDCl₃) δ: 1.35 (3H, s), 1.62 (3H, s), 2.80 (3H, d, J=5.1 Hz), 3.41 (1H, d, J=15.4 Hz), 3.64 (1H, d, J=10.6 Hz), 3.90-3.94 (2H, m), 4.24 (1H, d, J=5.3 Hz), 4.45-4.55 (4H, m), 4.67 (1H, br s), 4.75 (1H, d, J=11.6 Hz), 5.82 (1H, d, J=3.5 Hz), 7.24-7.33 (10H, m).

### Step 4 and Step 5 Synthesis of Compound 6

At room temperature, the crude product of compound 4 (51 mg) was dissolved in 0.1% (v/v) solution of concentrated sulfuric acid and acetic acid (1.5 mL). To the solution was added anhydrous acetic acid (121 μL, 1.28 mmol), and the mixture was stirred for 2 hours. The reaction solution was poured into a mixture of saturated sodium bicarbonate water and ethyl acetate, and then the organic layer was separated. The aqueous layer was extracted with ethyl acetate, and the organic layer was washed with water and brine. After drying over sodium sulfate, the solvent was evaporated under reduced pressure to give the crude product of compound 5 (65 mg). At room temperature, to a solution of the crude product of compound 5 (64 mg) in acetonitrile (1.0 mL) were added silylated thymine (46 mg, 0.170 mmol) and trimethylsilyl trifluoromethanesulfonate (20 μL, 0.113 mmol), and the mixture was stirred at 90° C. for 2 hours. To the reaction solution was added saturated sodium bicarbonate water, and the mixture was extracted with ethyl acetate. After the organic layer was washed with water and brine, it was dried over sodium sulfate, and the solvent was evaporated under reduced pressure. The resultant crude product was purified by silica gel column chromatography (n-hexane:ethyl acetate=60:40→30:70) to give compound 6 (50 mg, 74% (overall yield from Step 3 to Step 5)) as white solid.

¹H-NMR (CDCl₃) δ: 1.57 (3H, s), 2.14 (3H, s), 2.33 (3H, s), 3.20 (3H, s), 3.42 (1H, d, J=15.4 Hz), 3.65 (1H, d, J=10.1 Hz), 4.14 (1H, d, J=10.1 Hz), 4.33 (1H, d, J=15.4 Hz), 4.43-4.48 (2H, m), 4.53-4.63 (3H, m), 5.45 (1H, br s), 5.97 (1H, d, J=2.3 Hz), 7.20 (1H, s), 7.26-7.35 (10H, m), 8.00 (1H, s).

### Step 6 Synthesis of Compound 7

On ice-cooling, to a solution of compound 6 (47 mg, 0.075 mmol) in tetrahydrofuran (0.47 mL) was added 40% methylamine aqueous solution (0.24 mL), and the mixture was stirred on ice-cooling for 35 minutes. After tetrahydrofuran was evaporated under reduced pressure, to the residue was added water, and the mixture was extracted with ethyl acetate. The organic layer was washed with water and brine, and then dried over sodium sulfate. The solvent was evaporated under reduced pressure to give the crude product of compound 7 (42 mg).

¹H-NMR (CDCl₃) δ: 1.57 (3H, s), 2.76 (3H, d, J=5.3 Hz), 2.76 (1H, br s), 3.20 (1H, d, J=15.2 Hz), 3.69 (1H, d, J=10.4 Hz), 3.87 (1H, d, J=15.2 Hz), 4.16 (1H, d, J=10.4 Hz), 4.32 (1H, d, J=5.8 Hz), 4.39 (1H, br s), 4.52-4.59 (3H, m), 4.64 (1H, d, J=11.4 Hz), 4.75 (1H, d, J=11.4 Hz), 5.92 (1H, d, J=4.8 Hz), 7.28-7.41 (11H, m), 8.44 (1H, br s).

### Step 7 Synthesis of Compound 8

At room temperature, to a solution of the crude product of compound 7 (42 mg) in pyridine (0.4 mL) was added methanesulfonyl chloride (14 μL, 0.180 mmol), and the mixture was stirred for 2 hours. To the reaction solution was added water, and the mixture was extracted with ethyl acetate. The organic layer was washed with 10% aqueous citric acid solution, saturated sodium bicarbonate water, water and brine. After drying over sodium sulfate, the solvent was evaporated under reduced pressure to give the crude product of compound 8 (46 mg).

¹H-NMR (CDCl₃) δ: 1.44 (3H, s), 2.73 (3H, d, J=5.3 Hz), 3.15 (3H, s), 3.26 (1H, d, J=15.1 Hz), 3.67-3.71 (2H, m), 4.23 (1H, d, J=10.5 Hz), 4.30 (1H, q, J=5.3 Hz), 4.48-4.56 (4H, m), 4.90 (1H, d, J=11.3 Hz)_{;} 5.38 (1H, dd, J=5.2, 4.2 Hz), 6.19 (1H, d, J=4.2 Hz), 7.24-7.40 (10H, m), 7.49 (1H, s), 8.19 (1H, s).

### Step 8 Synthesis of Compound 9

At room temperature, to a solution of the crude product of compound 8 (46 mg) in tetrahydrofuran-ethanol (3/2, 0.5 mL) was added 2 mol/L aqueous sodium hydroxide solution (0.15 mL, 0.300 mmol), and the mixture was stirred for 20 hours. On ice-cooling, to the reaction solution was added 2 mol/L hydrochloric acid solution to neutralize, and the solvent was evaporated under reduced pressure. To the residue was added water, and the mixture was extracted with ethyl acetate. The organic layer was washed with water and brine, and then dried over sodium sulfate. The solvent was evaporated under reduced pressure, and the resultant crude product was purified by silica gel column chromatography (n-hexane:ethyl acetate=50:50→20:80) to give compound 9 (28 mg, 68% (overall yield from Step 6 to Step 8)) as white solid.

¹H-NMR (CDCl₃) δ: 1.71 (3H, s), 2.66 (3H, d, J=5.1 Hz), 3.35 (1H, d, J=15.2 Hz), 3.61 (1H, d, J=15.2 Hz), 3.87 (1H, d, J=10.4 Hz), 4.17-4.29 (4H, m), 4.46 (1H, d, J=7.8 Hz), 4.53-4.65 (3H, m), 4.77 (1H, d, J=11.1 Hz), 6.16 (1H, d, J=2.3 Hz), 7.26-7.36 (16H, m), 7.44 (1H, s), 8.62 (1H, s).

### Step 9 Synthesis of Compound 10

Under nitrogen stream, to a solution of compound 9 (29 mg, 0.054 mmol) in pyridine (0.3 mL) was added trifluoromethanesulfonic anhydride (27 μL, 0.161 mmol) on ice-cooling-, and the mixture was stirred for 5 hours. Furthermore, after the mixture was stirred at room temperature for 2 hours, to the reaction solution was added water, and the mixture was extracted with ethyl acetate. The organic layer was washed with water and brine, and then dried over sodium sulfate. The solvent was evaporated under reduced pressure to give the crude product of compound 10 (39 mg).

¹H-NMR (CDCl₃) δ: 1.81 (3H, s), 2.68 (3H, d, J=5.1 Hz), 3.40 (1H, d, J=15.2 Hz), 3.68 (1H, d, J=15.2 Hz), 3.76 (1H, d, J=9.9 Hz), 3.87 (1H, d, J=9.9 Hz), 4.24 (1H, q, J=5.1 Hz), 4.52-4.59 (4H, m), 4.76 (1H, d, J=11.4 Hz), 5.46 (1H, dd, J=4.4, 2.6 Hz), 6.33 (1H, d, J=4.4 Hz), 7.20 (1H, s), 7.26-7.38 (10H, m), 8.05 (1H, s).

### Step 10 Synthesis of Compound 11

Under nitrogen stream, to a solution of the crude product of compound 10 (39 mg) in acetonitrile (0.7 mL) was added potassium carbonate (19 mg, 0.135 mmol) at room temperature, and the mixture was stirred at 40° C. for 5.5 hours. To the reaction solution was added water, and the mixture was extracted with ethyl acetate. The organic layer was washed with brine. After drying over sodium sulfate, the solvent was evaporated under reduced pressure. The resultant crude product was purified by silica gel column chromatography (n-hexane:ethyl acetate=70:30→40:60) to give compound 11 (24 mg, 86% (overall yield from Step 9 to Step 10)) as white solid.

¹H-NMR (CDCl₃) δ: 1.46 (3H, s), 2.99 (3H, s), 3.22 (1H, d, J=12.9 Hz), 3.52 (1H, d, J=10.6 Hz), 3.61 (1H, d, J=12.9 Hz), 3.71 (1H, d, J=3.8 Hz), 3.76 (1H, d, J=10.6 Hz), 4.13 (1H, d, J=3.8 Hz), 4.54-4.61 (3H, m), 4.66 (1H, d, J=11.4 Hz), 6.31 (1H, s), 7.24-7.37 (10H, m), 7.84 (1H, s), 8.16 (1H, br s).

### Step 11 Synthesis of Compound 12

To a solution of compound 11 (21 mg, 0.040 mmol) in tetrahydrofuran-methanol (1:1, 0.6 mL) was added 20% palladium hydroxide-carbon powder (5.3 mg), and the mixture was stirred under hydrogen stream at room temperature for 20 hours. The reaction solution was filtered, and then the solvent was evaporated to give compound 12 (17 mg) as white solid.

¹H-NMR (MeOD) δ: 1.86 (3H, s), 3.01 (3H, s), 3.29 (1H, d, J=13.2 Hz), 3.51 (1H, d, J=13.2 Hz), 3.66 (1H, d, J=12.3 Hz), 3.77 (1H, d, J=12.3 Hz), 3.81 (1H, d, J=4.5 Hz), 4.27 (1H, d, J=4.5 Hz).

### Step 12 Synthesis of Compound 13

Under nitrogen stream, to a solution of compound 12 (31 mg, 0.090 mmol) in pyridine (0.6 mL) was added 4,4′-dimethoxytrityl chloride (46 mg, 0.135 mmol) at room temperature, and the mixture was stirred for 15.5 hours. At room temperature, 4,4′-dimethoxytrityl chloride (46 mg, 0.135 mmol) was added thereto, and the mixture was stirred for 7.5 hours. Furthermore, 4,4′-dimethoxytrityl chloride (46 mg, 0.135 mmol) was added thereto at room temperature, and the mixture was stirred for 14.5 hours. Then, to the reaction solution was added saturated sodium bicarbonate water, and the mixture was extracted with ethyl acetate. The organic layer was washed with water and brine, and then dried over sodium sulfate. The solvent was evaporated under reduced pressure, and the resultant crude product was purified by silica gel column chromatography (chloroform:methanol=100:0→95:5) to give compound 13 (59 mg) as white solid.

¹H-NMR (CDCl₃) δ: 1.36 (3H, s), 2.82 (1H, d, J=7.7 Hz), 3.09 (3H, s), 3.33-3.41 (3H, m), 3.49 (1H, d, J=13.6 Hz), 3.80 (6H, s), 3.85 (1H, d, J=5.0 Hz), 4.44 (1H, dd, J=7.0, 5.0 Hz), 6.36 (1H, s), 6.85 (4H, d, J=8.0 Hz), 7.24-7.33 (5H, m), 7.40 (2H, d, J=7.2 Hz), 7.64 (1H, s), 8.28 (1H, br s).

### Step 13 Synthesis of Compound I-1

Under nitrogen stream, to a solution of compound 13 (59 mg, 0.091 mmol) in anhydrous dichloromethane (0.9 mL) were added diisopropylethylamine (63 μL, 0.363 mmol), 2-cyanoethyl-N,N-diisopropylchloro phosphoramidite (61 μL, 0.272 mmol), and the mixture was stirred for 3 hours. To the reaction solution was added saturated sodium bicarbonate water, and the mixture was extracted with ethyl acetate. The organic layer was washed with water and brine, and then dried over anhydrous sodium sulfate. The solvent was evaporated, and the resultant crude product was purified by silica gel column chromatography (n-hexane:ethyl acetate=34:66) to give compound I-1 (47 mg, 61%) as white solid.

³¹P-NMR (CDCl₃) δ_{P}: 149.2, 150.7.

### Example 1-2

### Synthesis of a Nucleotide of the Present Invention (Compound I-2)

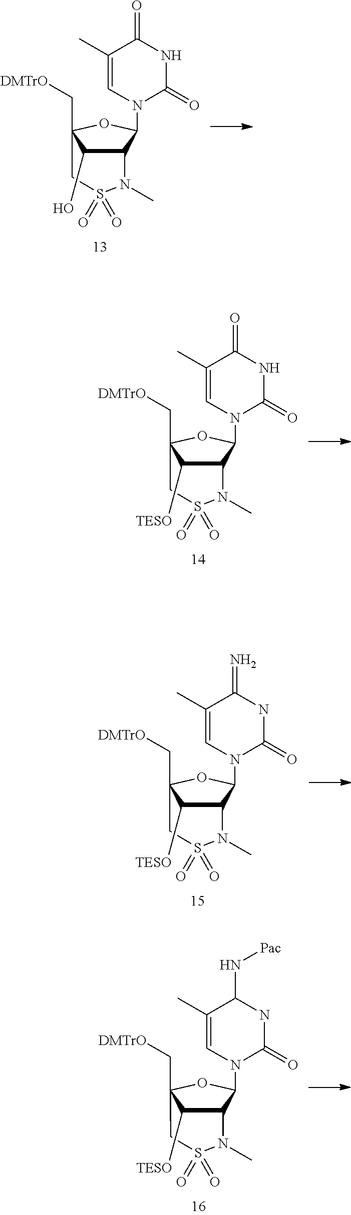

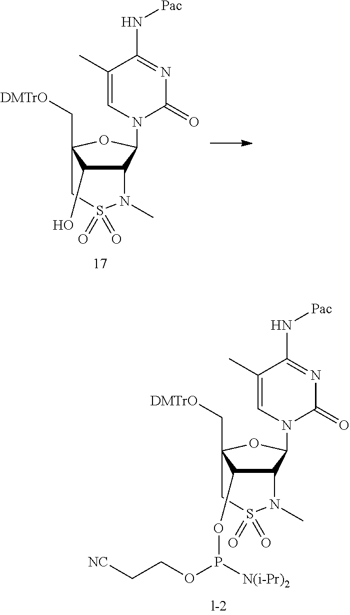

### Step 1 Synthesis of Compound 14

Under nitrogen stream, to a solution of compound 13 synthesized in Example 1-1 (191 mg, 0.294 mmol) in N,N-dimethylformamide (1.0 mL) were added imidazole (160 mg, 2.35 mmol) and chlorotriethylsilane (199 μL, 1.18 mmol) at room temperature, and the mixture was stirred for 3 hours. To the reaction solution was added water, and the mixture was extracted with ethyl acetate. The organic layer was washed with water and brine, and then dried over sodium sulfate. The solvent was evaporated under reduced pressure, and the resultant crude product was purified by silica gel column chromatography (n-hexane:ethyl acetate=75:25→60:50) to give compound 14 (214 mg, 95%) as white solid.

¹H-NMR (CDCl₃) δ: 0.48-0.63 (6H, m), 0.87 (9H, t, J=7.9 Hz), 1.23 (3H, s), 3.04 (3H, s), 3.15 (1H, d, J=10.4 Hz), 3.24 (1H, d, J=13.2 Hz), 3.45 (1H, d, J=10.4 Hz), 3.46 (1H, d, J=13.2 Hz), 3.70 (1H, d, J=4.4 Hz), 3.80 (3H, s), 3.80 (3H, s), 4.40 (1H, d, J=4.4 Hz), 6.32 (1H, s), 6.82-6.85 (4H, m), 7.23-7.32 (7H, m), 7.37 (2H, d, J=6.9 Hz), 7.86 (1H, s), 8.10 (1H, s).

### Step 2 Synthesis of Compound 15

Under nitrogen stream, to a solution of compound 14 (206 mg, 0.269 mmol) in acetonitrile (2.0 mL) were added triethylamine (149 μL, 1.08 mmol), N,N-dimethylaminopyridine (6.6 mg, 0.054 mmol) and 2,4,6-triisopropylbenzenesulfonyl chloride (122 mg, 0.403 mmol) at room temperature, and the mixture was stirred for 20 hours. At room temperature, to the reaction solution was added 28% ammonia water (2.0 mL), and the mixture was stirred for 2 hours. The solvent was evaporated under reduced pressure, and to the resultant residue was added water. The mixture was extracted with ethyl acetate. The organic layer was washed with water and brine, and then dried over sodium sulfate. The solvent was evaporated under reduced pressure, and the resultant crude product was purified by silica gel column chromatography (chloroform:methanol=100:0→95:5) to give compound 15 (180 mg, 88%) as white solid.

¹H-NMR (CDCl₃) δ: 0.45-0.60 (6H, m), 0.84 (9H, t, J=7.9 Hz), 1.22 (3H, s), 3.09 (3H, s), 3.13 (1H, d, J=10.4 Hz), 3.21 (1H, d, J=13.2 Hz), 3.45-3.48 (2H, m), 3.80 (3H, s), 3.81 (3H, s), 3.85 (1H, d, J=4.4 Hz), 4.36 (1H, d, J=4.4 Hz), 6.34 (1H, s), 6.82-6.85 (4H, m), 7.24-7.32 (7H, m), 7.39 (2H, d, J=6.9 Hz), 7.96 (1H, s).

### Step 3 and Step 4 Synthesis of Compound 17

On ice-cooling, to a solution of phenoxyacetyl chloride (38 μL, 0.276 mmol) in dichloromethane (2.1 mL) was added 1-methylimidazole (25 μL, 0.313 mmol), and the mixture was stirred for 10 minutes. The resultant suspension was added to a solution of compound 15 in pyridine (1.4 mL), and the mixture was stirred for 1.5 hours. To the reaction solution was added saturated sodium bicarbonate water, and the mixture was extracted with ethyl acetate. The organic layer was washed with water and brine, and then dried over sodium sulfate. The solvent was evaporated under reduced pressure to give the crude product of compound 16 (171 mg). At room temperature, to a solution of the crude product of compound 16 in tetrahydrofuran (2.1 mL) was added a solution of 1M tetra-n-butylammonium fluoride in tetrahydrofuran (221 μL, 0.221 mmol), and the mixture was stirred for 15 minutes. To the reaction solution was added ethyl acetate, and then the solvent was evaporated. The resultant crude product was purified by silica gel column chromatography (n-hexane:ethyl acetate=60:40→15:85) to give compound 17 (77 mg, 53%) as white solid.

¹H-NMR (CDCl₃) δ: 1.39 (3H, s), 3.09 (4H, br s), 3.33-3.42 (3H, m), 3.50 (1H, d, J=13.6 Hz), 3.80 (3H, s), 3.81 (3H, s), 3.95 (1H, s), 4.46 (1H, d, J=4.8 Hz), 4.84 (2H, br s), 6.39 (1H, s), 6.84-6.87 (4H, m), 6.93-7.01 (3H, m), 7.24-7.33 (9H, m), 7.40 (2H, d, J=7.3 Hz), 7.91 and 8.06 (1H, br s).

### Step 5 Synthesis of Compound I-2

Under nitrogen stream, to a solution of compound 17 (100 mg, 0.127 mmol) in anhydrous dichloromethane (1.5 mL) were added diisopropylethylamine (89 μL, 0.509 mmol) and 2-cyanoethyl-N,N-diisopropylchloro phosphoramidite (85 μL, 0.382 mmol), and the mixture was stirred for 2.5 hours. To the reaction solution was added saturated sodium bicarbonate water, and the mixture was extracted with ethyl acetate. The organic layer was washed with water and brine, and then dried over anhydrous sodium sulfate. The solvent was evaporated, and the resultant crude product was purified by silica gel column chromatography (n-hexane:ethyl acetate=60:40→35:65) to give compound I-2 (69 mg, 55%) as white solid.

³¹P-NMR (CDCl₃) δ_{P}: 149.1, 150.9.

### Example 1-3

### Synthesis of the Nucleotide of the Present Invention (Compound III-4)

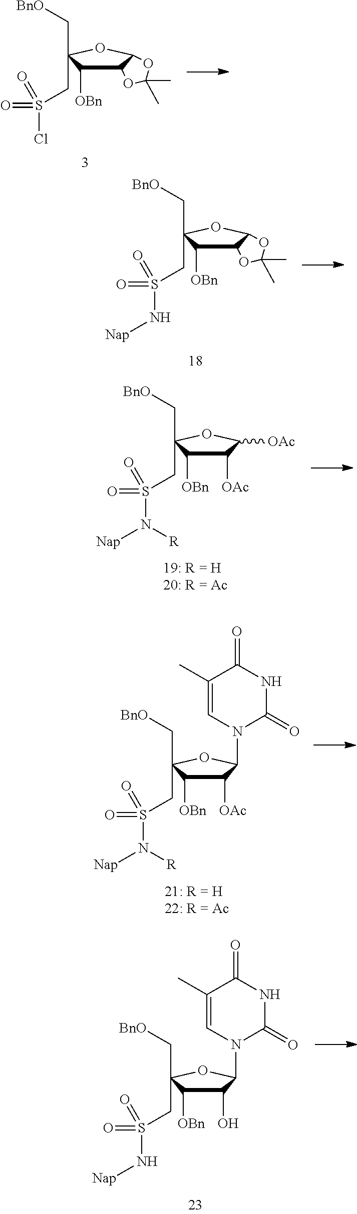

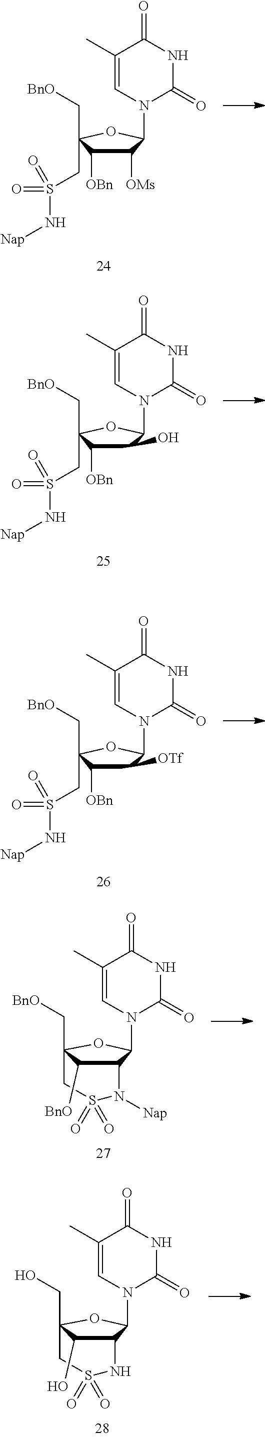

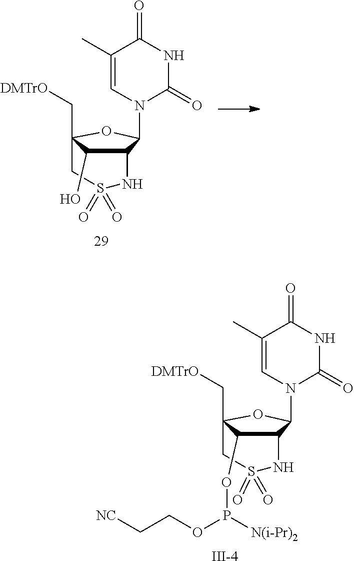

### Step 1 Synthesis of Compound 18

Under nitrogen stream, to a solution of compound 3 (16.8 g, 34.8 mmol) in tetrahydrofuran (120 mL) were added triethylamine (12.1 mL, 87.0 mmol) and 2-naphthylmethylamine hydrochloride (J. Carbohydr. Chem. 30, 559-574 (2011)) (7.41 g, 38.3 mmol) on ice-cooling, and the mixture was stirred for 1 hour. After the solvent was evaporated under reduced pressure, to the residue was added water, and the mixture was extracted with ethyl acetate. The organic layer was washed with water and brine, and then dried over sodium sulfate. The resultant crude product was purified by silica gel column chromatography (n-hexane:ethyl acetate=90:10→60:40) to give compound 18 (16.6 g, 79° A) as light yellow foam.

¹H-NMR (CDCl₃) δ: 1.37 (3H, s), 1.65 (3H, s), 3.54 (1H, d, J=15.6 Hz), 3.68 (1H, d, J=10.8 Hz), 3.95 (1H, d, J=10.8 Hz), 4.04 (1H, d, J=15.6 Hz), 4.27 (1H, d, J=5.6 Hz), 4.44-4.55 (5H, m), 4.69 (1H, dd, J=5.6, 4.0 Hz), 4.75 (1H, d, J=11.6 Hz), 4.94 (1H, dd, J=6.8, 5.6 Hz), 5.85 (1H, d, J=4.0 Hz), 7.23-7.35 (10H, m), 7.44-7.49 (3H, m), 7.79-7.83 (4H, m).

### Step 2 and Step 3 Synthesis of Compounds 21 and 22

To a solution of compound 18 (16.6 g, 27.4 mmol) in acetic acid (80 mL) were added concentrated sulfuric acid (80 μL, 1.44 mmol) and anhydrous acetic acid (31.1 mL, 329 mmol) at room temperature, and the mixture was stirred for 4 hours. The reaction solution was poured into saturated sodium bicarbonate water, and the mixture was extracted with ethyl acetate. The organic layer was washed with water and brine, and then dried over magnesium sulfate. The solvent was evaporated under reduced pressure to give a mixture of compounds 19 and 20 (18.3 g). Under nitrogen stream, to a solution of the mixture of compounds 19 and 20 (18.3 g) in acetonitrile (150 mL) were added thymine (5.18 g, 41.1 mmol) and N,O-bis(trimethylsilyl)acetamide (23.5 mL, 96.0 mmol) at room temperature, and the mixture was stirred at 40° C. for 20 minutes. At room temperature, trimethylsilyl trifluoromethanesulfonate (4.95 mL, 27.4 mmol) was added thereto, and the mixture was heated to reflux for 5 hours. To the reaction solution was added saturated sodium bicarbonate water, and the mixture was extracted with ethyl acetate. The organic layer was washed with water and brine, and then dried over magnesium sulfate. The solvent was evaporated under reduced pressure. The resultant crude product was purified by silica gel column chromatography (n-hexane:ethyl acetate=70:30→45:55) to give a mixture of compounds 21 and 22 (18.6 g) as white solid.

### Step 4 Synthesis of Compound 23

To a solution of the mixture of compounds 21 and 22 (18.6 g) in tetrahydrofuran (90 mL) was added 40% methylamine aqueous solution (45 mL) on ice-cooling, and the mixture was stirred for 40 minutes. After tetrahydrofuran was evaporated under reduced pressure, to the residue was added water, and the mixture was extracted with ethyl acetate/methanol (5/1). The organic layer was washed with water and brine, and then dried over magnesium sulfate. The solvent was evaporated under reduced pressure to give the crude product of compound 23 (16.7 g).

¹H-NMR (CDCl₃) δ: 1.53 (3H, s), 2.80 (2H, d, J=4.8 Hz), 3.13 (1H, d, J=15.2 Hz), 3.62 (1H, d, J=10.4 Hz), 3.69 (1H, d, J=15.2 Hz), 4.08 (1H, d, J=10.4 Hz), 4.19 (1H, d, J=5.6 Hz), 4.34-4.56 (6H, m), 5.15 (111, br s), 5.88 (1H, d, J≦5.2 Hz), 7.18-7.35 (11H, m), 7.44-7.49 (3H, m), 7.80-7.83 (4H, m), 8.22 (1H, br s).

### Step 5 Synthesis of Compound 24

Under nitrogen stream, to a solution of the crude product of compound 23 (16.7 g) in pyridine (80 mL) was added methanesulfonyl chloride (4.79 mL, 61.5 mmol) at room temperature, and the mixture was stirred for 1.5 hours. After the solvent was evaporated under reduced pressure, to the residue was added water, and the mixture was extracted with ethyl acetate. The organic layer was washed with water and brine, and then dried over magnesium sulfate. The solvent was evaporated under reduced pressure to give the crude product of compound 24 (18.7 g).

¹H-NMR (CDCl₃) δ: 1.42 (3H, s), 3.08 (3H, s), 3.25 (1H, d, J=15.2 Hz), 3.62-3.70 (2H, m), 4.19 (1H, d, J=10.4 Hz), 4.33-4.53 (6H, m), 4.76-4.82 (2H, m), 5.37 (1H, t, J=4.8 Hz), 6.16 (1H, d, J=4.0 Hz), 7.23-7.40 (12H, m), 7.48-7.50 (2H, m), 7.73 (1H, s), 7.82-7.84 (3H, m), 8.13 (1H, br 5).

### Step 6 Synthesis of Compound 25

To a solution of the crude product of compound 24 (18.7 g) in tetrahydrofuran-ethanol (3/2, 150 mL) was added 2 n-mol/L aqueous sodium hydroxide solution (61.5 mL, 123 mmol) at room temperature, and the mixture was stirred for 22 hours. On ice-cooling, to the reaction solution was added concentrated hydrochloric acid (10.2 mL, 123 mmol) to neutralize, and then saturated sodium bicarbonate water was added thereto. Tetrahydrofuran and ethanol was evaporated under reduced pressure. To the residue was added water, and the mixture was extracted with ethyl acetate. The organic layer was washed with water and brine, and then dried over magnesium sulfate. The solvent was evaporated under reduced pressure, and the resultant crude product was purified by silica gel column chromatography (n-hexane:ethyl acetate=60:40→30:70) to give compound 25 (11.9 g, 64% (overall yield from Step 2 to Step 6)) as white solid.

¹H-NMR (CDCl₃) δ: 1.62 (3H, s), 3.33 (1H, d, J=15.2 Hz), 3.58 (1H, d, J=15.2 Hz), 3.82 (1H, d, J=10.0 Hz), 4.11 (1H, d, J=10.0 Hz), 4.14-4.20 (2H, m), 4.29 (1H, dd, J=14.8, 5.6 Hz), 4.32-4.39 (2H, m), 4.42 (1H, d, J=11.6 Hz), 4.53 (1H, d, J=11.2 Hz), 4.58 (1H, d, J=11.6 Hz), 4.62 (1H, d, J=11.2 Hz), 4.88 (1H, t, J=6.0 Hz), 6.14 (1H, d, J=4.0 Hz), 7.21-7.35 (12H, m), 7.45-7.48 (2H, m), 7.64 (1H, s), 7.76-7.81 (3H, m), 8.83 (1H, s).

### Step 7 and Step 8 Synthesis of Compound 27

Under nitrogen stream, to a solution of compound 25 (11.9 g, 17.7 mmol) in pyridine (60 mL) was added trifluoromethanesulfonic anhydride (8.95 mL, 53.0 mmol) on ice-cooling, and the mixture was stirred for 15 hours. To the reaction solution was added water, and the mixture was extracted with ethyl acetate. The organic layer was washed with water and brine, and then dried over magnesium sulfate. The solvent was evaporated under reduced pressure to give the crude product of compound 26 (22.6 g). Under nitrogen stream, to a solution of the crude product of compound 26 (22.6 g) in acetonitrile (220 mL) was added potassium carbonate (6.10 g, 44.2 mmol) at room temperature, and the mixture was stirred at 40° C. for 4.5 hours. To the reaction solution was added water, and acetonitrile was evaporated under reduced pressure. The residue was extracted with ethyl acetate, and the organic layer washed with water and brine. After drying over magnesium sulfate, the solvent was evaporated under reduced pressure, and the resultant crude product was purified by silica gel column chromatography (n-hexane:ethyl acetate=65:35→50:50) to give compound 27 (8.73 g, 76%) as white solid.

¹H-NMR (CDCl₃) δ: 1.39 (3H, s), 3.33 (1H, d, J=12.8 Hz), 3.53 (1H, d, J=10.8 Hz), 3.77 (1H, d, J=12.8 Hz), 3.80 (1H, d, J=10.8 Hz), 3.89 (1H, d, J=4.4 Hz), 3.92 (1H, d, J=11.6 Hz), 3.98 (1H, d, J=4.4 Hz), 4.02 (1H, d, J=11.6 Hz), 4.37 (1H, d, J=14.0 Hz), 4.52 (1H, d, J=11.6 Hz), 4.57 (1H, d, J=11.6 Hz), 5.07 (1H, d, J=14.0 Hz), 6.51 (1H, s), 6.98-7.00 (2H, m), 7.18-7.20 (2H, m), 7.23-7.30 (6H, m), 7.43-7.48 (2H, m), 7.58 (1H, dd, J=8.4, 1.6 Hz), 7.69-7.73 (2H, m), 7.76-7.80 (2H, m), 7.95 (1H, s), 8.35 (1H, s).

### Step 9 Synthesis of Compound 28

Under nitrogen stream, to a solution of compound 27 (4.00 g, 6.12 mmol) in dichloromethane (40 mL) was added dropwise boron trichloride (1 mol/L dichloromethane solution, 61.2 mL, 61.2 mmol) at −78° C., and the mixture was gradually warmed to room temperature and stirred for 40 hours. To the reaction solution was added dropwise methanol (40 mL) at −78° C., and the mixture was stirred for 20 minutes at room temperature. The solvent was evaporated under reduced pressure, and the resultant crude product was purified by silica gel column chromatography (chloroform:methanol=96:4→85:15) to give compound 28 (1.59 g, 78%) as white solid.

¹H-NMR (MeOD) δ: 1.86 (3H, s), 3.20 (1H, d, J=13.2 Hz), 3.43 (1H, d, J=13.2 Hz), 3.66 (1H, d, J=12.4 Hz), 3.75 (1H, d, J=12.4 Hz), 3.78 (1H, d, J=4.0 Hz), 4.24 (1H, d, J=4.0 Hz), 6.34 (1H, s), 8.29 (1H, s).

### Step 10 Synthesis of Compound 29

Under nitrogen stream, to a solution of compound 28 (1.76 g, 5.28 mmol) in pyridine (18 mL) was added 4,4′-dimethoxytrityl chloride (3.04 g, 8.98 mmol) at room temperature, and the mixture was stirred for 15 hours. After the solvent was evaporated under reduced pressure, to the residue was added saturated sodium bicarbonate water, and the mixture was extracted with ethyl acetate. The organic layer was washed with water and brine, and then dried over sodium sulfate. The solvent was evaporated under reduced pressure, and the resultant crude product was purified by silica gel column chromatography (chloroform:methanol=100:0→94:6) to give compound 29 (3.30 g, 98%) as white solid.

¹H-NMR (CDCl₃) δ: 1.37 (3H, s), 3.33-3.43 (3H, m), 3.52 (1H, d, J=13.2 Hz), 3.72 (3H, s), 3.73 (3H, s), 4.15 (1H, s), 4.29 (1H, br s), 4.38 (1H, s), 6.04 (1H, br s), 6.17 (1H, s), 6.81 (4H, d, J=8.8 Hz), 7.17 (1H, t, J=7.2 Hz), 7.24-7.30 (6H, m), 7.41 (2H, d, J=7.6 Hz), 7.49 (1H, s), 9.90 (1H, br s).

### Step 11 Synthesis of Compound III-4

Under nitrogen stream, to a solution of compound 29 (21 mg, 0.033 mmol) in acetonitrile (0.4 mL) were added 2-cyanoethyl-N,N,N′,N′-tetraisopropyl phosphordiamidite (21 μL, 0.065 mmol) and 5-ethylthio-1H-tetrazole (6.4 mg, 0.049 mmol) at room temperature, and the mixture was stirred for 7 hours. To the reaction solution was added saturated sodium bicarbonate water, and the mixture was extracted with ethyl acetate. The organic layer was washed with water and brine, and then dried over anhydrous sodium sulfate. The solvent was evaporated under reduced pressure, and the resultant crude product was purified by silica gel column chromatography (n-hexane:ethyl acetate=50:50→25:75) to give compound III-4 (18 mg, 65%) as white solid.

³¹P-NMR (CDCl₃) δ_{P}: 148.7, 151.9.

Similarly, the following nucleotides can be synthesized.

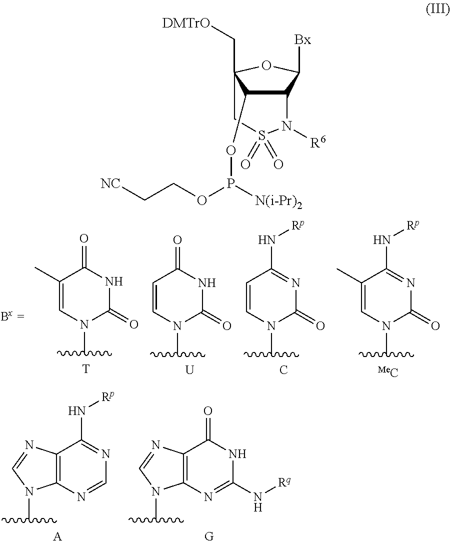

wherein R^{p} is acetyl, benzoyl or phenoxyacetyl, and R^{q} is isobutyl, acetyl, benzoyl or phenoxyacetyl.

TABLE 1

| Compound | Bx | R⁶ |
|---|---|---|
| III-1 | ^{Me}C | Me |
| III-2 | A | Me |
| III-3 | G | Me |
| III-5 | ^{Me}C | H |
| III-6 | A | H |
| III-7 | G | H |
| III-8 | T | Et |
| III-9 | ^{Me}C | Et |
| III-10 | A | Et |
| III-11 | G | Et |
| III-12 | T | i-Pr |
| III-13 | ^{Me}C | i-Pr |
| III-14 | A | i-Pr |
| III-15 | G | i-Pr |

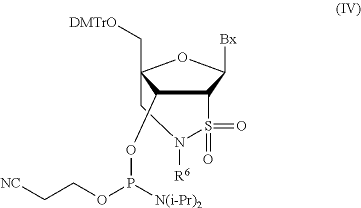

TABLE 2

| Compound | Bx | R⁶ |
|---|---|---|
| IV-1 | T | Me |
| IV-2 | ^{Me}C | Me |
| IV-3 | A | Me |
| IV-4 | G | Me |
| IV-5 | T | H |
| IV-6 | ^{Me}C | H |
| IV-7 | A | H |
| IV-8 | G | H |
| IV-9 | T | Et |
| IV-10 | ^{Me}C | Et |
| IV-11 | A | Et |
| IV-12 | G | Et |
| IV-13 | T | i-Pr |
| IV-14 | ^{Me}C | i-Pr |
| IV-15 | A | i-Pr |
| IV-16 | G | i-Pr |

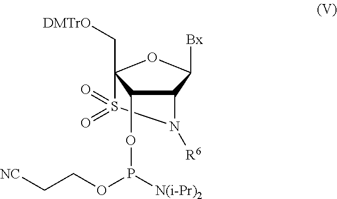

TABLE 3

| Compound | Bx | R⁶ |
|---|---|---|
| V-1 | T | Me |
| V-2 | ^{Me}C | Me |
| V-3 | A | Me |
| V-4 | G | Me |
| V-5 | T | H |
| V-6 | ^{Me}C | H |
| V-7 | A | H |
| V-8 | G | H |
| V-9 | T | Et |
| V-10 | ^{Me}C | Et |
| V-11 | A | Et |
| V-12 | G | Et |
| V-13 | T | i-Pr |
| V-14 | ^{Me}C | i-Pr |
| V-15 | A | i-Pr |
| V-16 | G | i-Pr |

### Example 2

### Synthesis of the Oligonucleotides of the Present Invention

The oligonucleotides (1) to (5) (Table 4) prepared with compound I-1 obtained in Example 1 and the oligonucleotides (6) and (7) (Table 5) prepared with compound III-4 obtained in Example 1 were synthesized by nS-8 (GeneDesign, Inc.) on the 0.2 μmol scale. Compound I-1 or III-4 (an amidite unit) was dissolved in acetonitrile to use. In Table 4, a nucleoside structure of compound I-1 (the following formula II-1) is indicated by Xa. In Table 5, a nucleoside structure of compound III-4 (the following formula II-2) is indicated by Xb. Duration of the coupling reaction between an amidite unit (compound I-1 or III-4) and a hydroxyl group at 5′-terminus was extended from 32 seconds (standard condition) to 16 minutes. The oligonucleotide with the 5′-terminus protected with a DMTr group supported on a solid phase was treated with 28% ammonia water: 40% methylamine aqueous solution (1:1), and the solvent was evaporated. The resultant crude product was partially purified by Sep-Pak C18 Plus Short Cartridge (Waters), and then purified by reversed-phase HPLC (Gilson PLC2020, using WatersXBridge™ Shield RP18 Column 5.0 μm (10 mm×50 mm) as a preparative column for the oligonucleotides (1) to (5); YMC Hydrosphere C18 Column 5.0 μm (10 mm×150 mm) for the oligonucleotides (6) and (7)).

The purities of the synthesized oligonucleotides were determined by reversed-phase HPLC, using WatersXBridge™ C18 Column 5.0 μm (4.6 mm×50 mm) for the oligonucleotides (1) to (5) (condition: gradient 6→10% (v/v) acetonitrile in 0.1 M triethyl ammonium acetate buffer (pH 7.0), 1 mL/min for 30 minutes) and YMC Hydrosphere C18 Column 5.0 μm (4.6 mm×100 mm) for the oligonucleotides (6) and (7) (condition: gradient 6→10% (v/v) acetonitrile in 0.01 M triethyl ammonium acetate buffer (pH 7.0), 1 mL/min for 30 minutes). The molecular weights were determined by MALDI-TOF-MASS (the oligonucleotides (1) to (5)) or ESI-TOF-MASS (the oligonucleotides (6) and (7)). The results are shown in Table 4 and Table 5.

TABLE 4

| | | MALDI-TOF-MASS | |
|---|---|---|---|
| | | Calculated | Found |
| Oligonucleotide | | (M+H⁺) | (M+H⁺) |
| 5′-d(GCG TTXa TTT GCT)-3′ | (1) | 3739.49 | 3739.04 |
| 5′-d(GCG TTXa TXaT GCT)-3′ | (2) | 3844.61 | 3845.25 |
| 5′-d(GCG XaTXa TXaT GCT)-3′ | (3) | 3949.72 | 3949.71 |
| 5′-d(GCG TTXa XaXaT GCT)-3′ | (4) | 3949.72 | 3950.25 |
| 5′-d(TTT TTT TTXa T)-3′ | (5) | 3086.09 | 3085.41 |

TABLE 5

| | | ESI-TOF-MASS | |
|---|---|---|---|
| | | Calculated | Found |
| Oligonucleotide | | (M − H)⁻ | (M − H)⁻ |
| 5′-d(GCGXbTXbTXbTGCT)-3′ | (6) | 3903.53 | 3904.43 |
| 5′-d(TTTTTTTTXbT)-3′ | (7) | 3068.47 | 3069.17 |

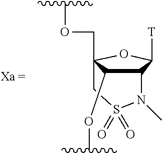

(II-1) 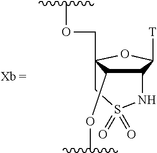
(II-2)

### Example 3

### Determination of the Melting Temperature (Tm) of the Oligonucleotides of the Present Invention

After the oligonucleotides (1) to (4) and (6) (antisense strands), which were the oligonucleotides synthesized in Example 2, and the sense strand (3′-CGC AAA AAA CGA-5′) were subjected to an annealing treatment, their Tm values were measured to determine the hybridization ability of the oligonucleotides (1) to (4) and (6). The nucleotide (0), which nucleoside moieties of the oligonucleotide are unmodified, is used as a control.

The sample solution (150 μM) containing 100 mM NaCl, 10 mM sodium phosphate buffer (pH 7.2), 4.0 μM oligonucleotide (antisense strand) and 4.0 μM sense strand was heated in heating blocks (95° C.) for 5 minutes, and then cooled to room temperature over 12 hours. Nitrogen stream was passed through the cell chamber of the spectrophotometer (SHIMADZU UV-1800) to prevent dew condensation, and the sample solution was gradually cooled to 15° C. and kept at 15° C. for 15 minutes before starting the measurements. The temperature was raised to 90° C. at the rate of 0.5° C./min while ultraviolet absorption spectra were measured at 260 nm at intervals of 0.5° C. Lidded cells were used to prevent concentration change due to rising temperature. The results are-shown in Table 6.

TABLE 6

| | | Sense strand | |
|---|---|---|---|
| | | RNA | DNA |
| Oligonucleotide | | complementary strand | complementary strand |
| (Antisense strand) | | Tₘ (ΔTₘ/mod.) (°C.) | Tₘ (ΔTₘ/mod.) (°C.) |
| 5′-d(GCG TTT TTT GCT)-3′ | (0) | 49.3 | 53.1 |
| 5′-d(GCG TTXa TTT GCT)-3′ | (1) | 49.7 (0.4) | 49.2 (-3.9) |
| 5′-d(GCG TTXa TXaT GCT)-3′ | (2) | 52.6 (1.7) | 46.3 (-3.4) |
| 5′-d(GCG XaTXa TXaT GCT)-3′ | (3) | 59.6 (3.4) | 46.6 (-2.2) |
| 5′-d(GCG TTXa XaXaT GCT)-3′ | (4) | 56.2 (2.3) | 47.4 (-1.9) |
| 5′-d(GCG XbTXb TXbT GCT)-3′ | (6) | 55.2 (2.0) | 42.0 (-3.7) |

As shown in Table 6, the Tm values of the oligonucleotides of the present invention hybridized to the RNA complementary strand are higher than that of the natural oligonucleotide hybridized to the RNA complementary strand. On the other hand, the Tm values of the oligonucleotides of the present invention hybridized to the DNA complementary strand are lower than that of the natural oligonucleotide hybridized to the DNA complementary strand. In addition, the higher the rate of the nucleoside structure (II-1) of the present invention comprised in the oligonucleotide, the higher the Tm value it has. Therefore, oligonucleotides prepared with a nucleotide(s) of the present invention have high affinities to single-stranded RNA, and they are easy to act on mRNA. Furthermore, they have low affinities to single-strand DNA, and therefore, their effects on DNA replication are little and concern of the toxicity is low. Thus, the oligonucleotides of the invention are useful as materials for synthesizing nucleic acid pharmaceuticals.

### Example 4

### Assessment of Nuclease Resistance of the Oligonucleotides of the Present Invention

The oligonucleotide (5) (Sulfonamide-NMe) and the oligonucleotide (7) (Sulfonamide-NH) synthesized in Example 2 were subjected to a test for determining the resistance to an exonuclease, which degrades an oligonucleotide from the 3′ terminus. The nucleotide (nature) wherein the Xa part of the oligonucleotide (5) was unmodified, the nucleotide (LNA) wherein the Xa part of the oligonucleotide (5) was the following 2′,4′-BNA/LNA, the nucleotide (AmNA (Amide-BNA)) wherein the Xa part of the oligonucleotide (5) was the following 2′,4′-BNA-amide, and the nucleotide (BNA^{NC}-Me) wherein the Xa part of the oligonucleotide (5) was the following 2′,4′-BNA^{NC}-Me, were used as a control.

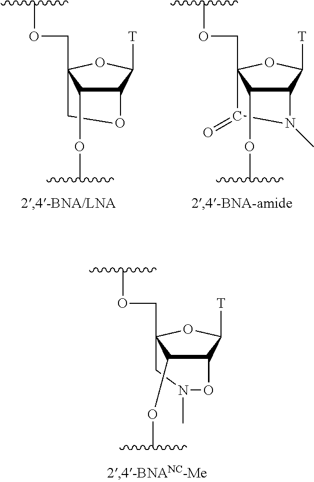

A buffer solution containing 750 pmol of the oligonucleotide (80 μL) was kept at 37° C. for 5 minutes, and then mixed with a buffer solution (20 μL) containing 0.4 μg phosphodiesterase I (Worthington Biochemical Corporation). Degradation of the oligonucleotide was determined over time by reverse HPLC (WatersXBridge™ Shield RP18 Column 2.5 μm (3.0 mm×50 mm)). The employed buffer contained 50 mM Tris HCl (pH 8.0) and 10 mM MgCl₂ (final concentration) and was sufficiently degassed before measurement. The condition of quantification by HPLC is as follows.

### (HPLC Quantification Condition)

Mobile phase:

Solution A: 0.1 M triethyl ammonium acetate buffer (pH 7.0)

Solution B: 0.1 M triethyl ammonium acetate buffer:acetonitrile=1:1 (v/v) (pH 7.0)

### Gradient:

The oligonucleotides (5) and (7): 16%-20% solution B (16 min)

The other oligonucleotides: 14%-22% solution B (16 min)

Column: WatersXBridge™ Shield RP18 Column 2.5 μm (3.0 mm×50 mm)
Flow rate: 0.8 mL/min
Column temperature: 50° C.

### Detection: UV (254 nm)

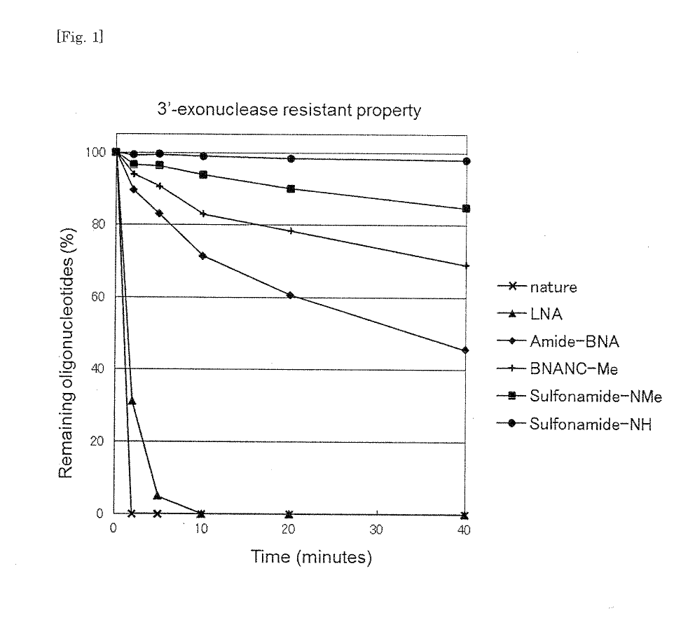

The result was shown in FIG. 1. In FIG. 1, “Remaining oligonucleotides (%)” refers to the ratio of the undegraded oligonucleotides (10mer) at the time of measurement to the undegraded oligonucleotides (10mer) at the time 0.

As shown, the nucleotide (nature) and the nucleotide (LNA) are degraded completely in 10 minutes. The remaining ratio of the nucleotide (AmNA(Amide-BNA)) after 40 minutes is 50% or less, and the remaining ratio of the nucleotide (BNA^{NC}-Me) after 40 minutes is 70% or less, while the remaining ratio of the oligonucleotide (5) (Sulfonamide-NMe) is 80% or more even after 40 minutes. In addition, the remaining ratio of the oligonucleotide (7) (Sulfonamide-NH) is 95% or more even after 40 minutes. Therefore, the oligonucleotides of the present invention having a sulfonamide structure have much higher enzyme-resistance than that of the oligonucleotides prepared with the unmodified oligonucleotide or the publicly known artificial nucleotides (LNA, AmNA (Amide-BNA) or BNA^{NC}-Me). Therefore, the oligonucleotides of the present invention have a very good in vivo persistence. Thus, the oligonucleotides of the present invention are useful as materials for synthesizing nucleic acid pharmaceuticals.

### INDUSTRIAL APPLICABILITY

As shown in the above Examples, oligonucleotides prepared with a nucleotide(s) or nucleoside(s) of the present invention show the superior binding affinity to a single strand RNA and nuclease resistance. Therefore, such oligonucleotides have a very good in vivo persistence. Thus, the nucleosides or nucleotides of the present invention are useful very much as materials for synthesizing nucleic acid pharmaceuticals such as antisense oligonucleotide and the like.

1. A compound of formula (I) or a salt thereof: 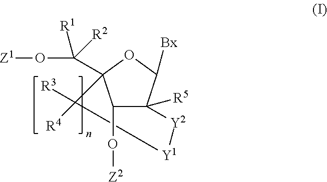
wherein
Y¹-Y² is S(═O)—NR⁶, S(═O)₂—NR⁶, NR⁶—S(═O) or NR⁶—S(═O)₂,
R⁶ is a hydrogen atom, substituted or unsubstituted alkyl, substituted or unsubstituted alkenyl, or substituted or unsubstituted alkynyl,
Bx is a nucleic acid base moiety,
Z¹ and Z² are each independently, a hydrogen atom, a hydroxyl protecting group or a reactive phosphorus group,
R¹ and R² are each independently, a hydrogen atom, halogen, cyano, substituted or unsubstituted alkyl, substituted or unsubstituted alkenyl, or substituted or unsubstituted alkynyl,
R³ are each independently, a hydrogen atom, halogen, cyano, substituted or unsubstituted alkyl, substituted or unsubstituted alkenyl, or substituted or unsubstituted alkynyl,
R⁴ are each independently, a hydrogen atom, halogen, cyano, substituted or unsubstituted alkyl, substituted or unsubstituted alkenyl, or substituted or unsubstituted alkynyl,
R⁵ is a hydrogen atom, halogen, cyano, substituted or unsubstituted alkyl, substituted or unsubstituted alkenyl, or substituted or unsubstituted alkynyl, and
n is an integer of 0 to 3.

2. The compound or salt thereof of claim 1, wherein Y¹-Y² is S(═O)₂—NR⁶, and R⁶ is a hydrogen atom or alkyl.

3. The compound or salt thereof of claim 1, wherein Bx is substituted or unsubstituted purin-9-yl, or substituted or unsubstituted 2-oxo-pyrimidin-1-yl.

4. The compound or salt thereof of claim 1, wherein Z¹ is a hydrogen atom or a hydroxyl protecting group.

5. The compound or salt thereof of claim 4, wherein the hydroxyl protecting group is acetyl, t-butyl, t-butoxymethyl, methoxymethyl, tetrahydropyranyl, 1-ethoxyethyl, 1-(2-chloroethoxyl)ethyl, 2-trimethylsilylethyl, p-chlorophenyl, 2,4-dinitrophenyl, benzyl, benzoyl, p-phenylbenzoyl, 2,6-dichlorobenzyl, levulinoyl, diphenylmethyl, p-nitrobenzyl, trimethylsilyl, triethylsilyl, t-butyldimethylsilyl, t-butyldiphenylsilyl, triphenylsilyl, triisopropylsilyl, benzoyl formate, chloroacetyl, trichloroacetyl, trifluoroacetyl, pivaloyl, isobutyryl, 9-fluorenylmethyloxycarbonyl, methansulfonyl, p-toluenesulfonyl, trifluoromethanesulfonyl, trityl, monomethoxytrityl, dimethoxytrityl, trimethoxytrityl, 9-phenylxanthin-9-yl or 9-(p-methoxyphenyl) xanthin-9-yl.

6. The compound or salt thereof of claim 1, wherein Z² is a hydrogen atom or a reactive phosphorus group.

7. The compound or salt thereof of claim 6, wherein the reactive phosphorus group is diisopropylcyanoethoxy phosphoramidite or H-phosphonate.

8. An oligonucleotide comprising one or more nucleoside structure of formula (II) or a pharmaceutically acceptable salt thereof: 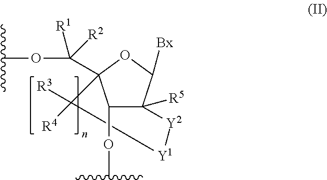
wherein
Y¹-Y² is S(═O)—NR⁶, S(═O)₂—NR⁶, NR⁶—S(═O) or NR⁶—S(═O)₂,
R⁶ is a hydrogen atom, substituted or unsubstituted alkyl, substituted or unsubstituted alkenyl, or substituted or unsubstituted alkynyl,
Bx is a nucleic acid base moiety,
R¹ and R² are each independently, a hydrogen atom, halogen, cyano, substituted or unsubstituted alkyl, substituted or unsubstituted alkenyl, or substituted or unsubstituted alkynyl,
R³ are each independently, a hydrogen atom, halogen, cyano, substituted or unsubstituted alkyl, substituted or unsubstituted alkenyl, or substituted or unsubstituted alkynyl,
R⁴ are each independently, a hydrogen atom, halogen, cyano, substituted or unsubstituted alkyl, substituted or unsubstituted alkenyl, or substituted or unsubstituted alkynyl,
R⁵ is a hydrogen atom, halogen, cyano, substituted or unsubstituted alkyl, substituted or unsubstituted alkenyl, or substituted or unsubstituted alkynyl, and
n is an integer of 0 to 3.

9. The compound or salt thereof of claim 2, wherein Bx is substituted or unsubstituted purin-9-yl, or substituted or unsubstituted 2-oxo-pyrimidin-1-yl.

10. The compound or salt thereof of claim 2, wherein Z¹ is a hydrogen atom or a hydroxyl protecting group.

11. The compound or salt thereof of claim 3, wherein Z¹ is a hydrogen atom or a hydroxyl protecting group.

12. The compound or salt thereof of claim 2, wherein Z² is a hydrogen atom or a reactive phosphorus group.

13. The compound or salt thereof of claim 3, wherein Z² is a hydrogen atom or a reactive phosphorus group.

14. The compound or salt thereof of claim 4, wherein Z² is a hydrogen atom or a reactive phosphorus group.

15. The compound or salt thereof of claim 5, wherein Z² is a hydrogen atom or a reactive phosphorus group.
